(19) United States
(12) Patent Application Publication
(10) Pub. No.: US 2018/0110784 A1
(43) Pub. Date: Apr. 26, 2018

(54) SYNTHETIC TLR4 AND TLR7 LIGANDS TO PREVENT, INHIBIT OR TREAT LIVER DISEASE

(71) Applicant: The Regents of the University of California, Oakland, CA (US)

(72) Inventors: Dennis A. Carson, La Jolla, CA (US); Tomoko Hayashi, San Diego, CA (US); Ekihiro Seki, San Diego, CA (US)

(21) Appl. No.: 15/565,097

(22) PCT Filed: Apr. 7, 2016
(86) PCT No.: PCT/US16/26522

§ 371 Date: Oct. 6, 2017

Related U.S. Application Data
(60) Provisional application No. 62/145,357, filed on Apr. 9, 2015.

(51) Int. Cl. A61K 31/52; A61P 1/16; A61K 47/60; A61K 9/00

(52) U.S. Cl. CPC A61K 31/52; A61P 1/16; A61K 9/0019; A61K 9/0043; A61K 9/0053; A61K 47/60

(57) ABSTRACT

Methods of using TLR7 conjugates or TLR4 ligands, or a combination thereof, to treat fibrosis are provided.

### CROSS-REFERENCE TO RELATED APPLICATIONS

This application claims the benefit of the filing date of U.S. application Ser. No. 62/145,357, filed Apr. 9, 2015, the disclosure of which is incorporated by reference herein.

### STATEMENT OF GOVERNMENT RIGHTS

The invention was made with government support under grants HHSN272200900034C and R01 AA020172 awarded by the National Institutes of Health. The Government has certain rights in the invention.

### BACKGROUND

All chronic liver diseases ultimately develop hepatic fibrosis/cirrhosis. Cirrhosis is the advanced form of liver fibrosis. Cirrhosis causes more than 32,000 deaths/year and is the 12^{th} leading cause of death in the U.S. The most common causes of hepatic fibrosis are chronic HBV or HCV infection, alcohol, non-alcoholic fatty liver disease (NAFLD), and biliary obstruction. Fibrosis/cirrhosis leads to portal hypertension, liver failure and hepatocellular carcinoma. There is currently no FDA approved anti-fibrotic therapy. Liver transplantation is the only cure for advanced liver disease.

Specifically, excessive alcohol consumption causes alcoholic steatosis, which may progress to alcoholic hepatitis (AH), fibrosis and ultimately cirrhosis and hepatocellular carcinoma (HCC). Alcohol-related liver death accounts for up to 44% of cirrhosis-associated deaths in the U.S. The six-month-mortality of severe alcoholic hepatitis (with cirrhosis) is 40%. Glucocorticoids have been used for the therapy of AH for 40 years but is associated with the many undesirable side effects that are well known in glucocorticoid therapy.

Nonalcoholic fatty liver disease (NAFLD) is one of the leading causes of liver disease in the U.S. The prevalence of obesity and diabetes is increasing: 60-70% of obese adults have NAFLD, 30-40% of adults in the western world have NAFLD, and 15-20% of NAFLD patients progress to nonalcoholic steatohepatitis (NASH). According to a 2008 estimation, NAFLD will be the leading cause for liver transplantation by 2020.

### SUMMARY

Toll-like Receptor (TLR) ligands, specifically TLR4 and TLR7 ligands, may help alter the progression of liver disease through a number of different mechanisms of action. The invention provides TLR4 ligands, such as compounds of formula (II), e.g., 1Z204, which signal predominately through the TRIF pathway and may act as a weak agonist, relative to 1Z65 or 1Z105, of the TLR4 signaling that can promote liver disease, e.g., liver fibrosis, alcoholic liver disease or non-alcoholic steatohepatitis. Thus, the use of certain TLR4 ligands may suppress the development or progression of liver disease, both alcoholic and non-alcoholic liver disease.

Also provided are TLR7 ligands and conjugates thereof, such compounds of formula (I), e.g., 1Z1, which may induce the TLR7 signaling pathway by inducing interferon-alpha, IL-10 and/or cross-tolerance for other TLRs, which are known to be protective for liver disease. Thus the use of certain TLR7 ligands and conjugates thereof may also suppress the development or progression of both alcoholic and non-alcoholic liver disease. Therefore, TLR7 ligands and conjugates as described herein, including compounds that are attenuated agonists relative to 1V136, are useful in the present methods.

Accordingly, the present invention provides uses for a synthetic TLR4 ligand, e.g., a weak TLR4 agonist, and/or a TLR7 ligand, e.g., an attenuated TLR7 agonist, optionally linked to an auxiliary group (a conjugate). In one embodiment, the synthetic TLR4 or TLR7 ligand, e.g., one having the auxiliary group, does not act as a prodrug. Such conjugates may include auxiliary groups that are linked to a TLR4 and/or TLR7 ligand via a linker, for instance, linked via an amino group, a carboxy group or a succinamide group.

In one embodiment, the invention provides methods of preventing, inhibiting or treating liver disease or fibrosis. In one embodiment, the method includes administering to a mammal in need thereof a composition having a TLR4 ligand and/or a TLR7 ligand or a conjugate thereof, in an amount effective to prevent, inhibit or treat liver disorders or fibrosis. In one embodiment, a single dose (which includes a TLR4 ligand, a TLR7 ligand, or a combination thereof) may show very potent activity in stimulating the immune response. Moreover, because of the low toxicity, in some circumstances higher doses may be administered, e.g., systemically, while under other circumstances lower doses may be administered. In one embodiment, the composition is locally administered, e.g., dermal or intranasal administration. In another embodiment, the composition is systemically administered.

Compounds disclosed herein may be useful in the development of small molecules that can 1) arrest or slow the progression of the disease process in both alcoholic and non-alcoholic liver disease; 2) serve as an effective therapy to allow for regeneration of the damaged liver tissues; and 3) help resolve underlying pathology in the case of liver disease caused by chronic HBV and HCV infections by inducing interferon-alpha which is known to be effective for treatment of these viral infections. The invention thus provides a composition comprising an amount of a synthetic TLR4 ligand, e.g., a compound of formula (II), and/or a composition comprising an amount of a synthetic TLR7 ligand, e.g., a compound of formula (I), or a composition comprising an amount of a synthetic TLR4 ligand and a synthetic TLR7 ligand, including a conjugate thereof, that is effective to prevent, inhibit or treat liver disease or fibrosis. In one embodiment, the composition does not include a solvent or preservative such as DMSO or ethanol, which may have toxic effects, e.g., in humans.

In one embodiment, the TLR7 ligand is a conjugate having formula (I):

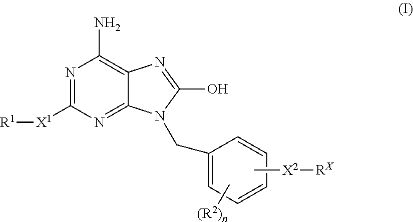

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, or substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₅)cycloalkyl, substituted (C₃-C₅)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R^{X} is an auxiliary group such as a macromolecule, e.g., a PEG moiety or derivative thereof;

or a pharmaceutically acceptable salt or solvate thereof.

In one embodiment, a compound of formula (I) has attenuated activity relative to 1V136:

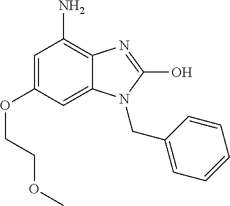 [SMILES: COCCOc1cc(N)c2nc(O)n(Cc3ccccc3)c2c1]

The auxiliary group can include organic molecules, composed of carbon, oxygen, hydrogen, nitrogen, sulfur, phosphorous, or combinations thereof, which are not harmful to body tissues (e.g., they are non-toxic, and/or do not cause inflammation) and may include, but not be limited to, dendrimers, proteins, peptides, lipids and their formulations (e.g., liposome nanoparticles), with or without linkers (X² groups), and amino-modified polymers, such as polystyrene beads, as well as α-galactosylceramides. Non-limiting examples of auxiliary groups include side chains that increase solubility, such as, for example, groups containing morpholino, piperidino, pyrrolidino, or piperazino rings and the like; amino acids, polymers of amino acids (proteins or peptides), e.g., dipeptides or tripeptides, and the like; carbohydrates (polysaccharides), nucleotides such as, for example, PNA, RNA and DNA, and the like; polymers of organic materials, such as, for example, polyethylene glycol, poly-lactide and the like; monomeric and polymeric lipids; insoluble organic nanoparticles; non-toxic body substances such as, for example, cells, lipids, vitamins, co-factors, antigens such as, for example, carbohydrates microbes, such as, for example, viruses, bacteria, fungi, and the like. The antigens can include inactivated whole organisms, or sub-components thereof and the like. A specific auxiliary group is an amino acid, a carbohydrate, a peptide, an antigen, a nucleic acid, a lipid, a dendrimer, a body substance, or a microbe. A specific peptide, has from 2 to about 20 amino acid residues. Another specific peptide, has from 10 to about 20 amino acid residues. A specific auxiliary group is a carbohydrate.

In one embodiment, R^{x} is ((R³)ᵣ—(R⁴)ₛ)ₚ or is R³. In one embodiment, R³ is a PEG moiety or a derivative of a PEG moiety. In certain embodiment R³ is —O—CH₂—CH₂— or —CH₂—CH₂—O—. In one embodiment, a PEG moiety can include one or more PEG units. A PEG moiety can include about 1 to about 1,000 PEG units, including, without limitation, about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800 or 900 units, in some embodiments. In certain embodiments, a PEG moiety can contain about 1 to 5 and up to about 25 PEG units, about 1 to 5 up to about 10 PEG units, about 10 up to about 50 PEG units, about 18 up to about 50 PEG units, about 47 up to about 150 PEG units, about 114 up to about 350 PEG units, about 271 up to about 550 PEG units, about 472 up to about 950 PEG units, about 50 up to about 150 PEG units, about 120 up to about 350 PEG units, about 250 up to about 550 PEG units or about 650 up to about 950 PEG units. A PEG unit is —O—CH₂—CH₂— or —CH₂—CH₂—O— in certain embodiments. In one embodiment, R⁴ is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic

In some embodiments R³ is —O—CH₂—CH₂— or —CH₂—CH₂—O— and r is about 1 to about 1000 (e.g., about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In some embodiments, r is about 5 to about 100, and sometimes r is about 5 to about 50 or about 5 to about 25. In certain embodiments, r is about 5 to about 15 and sometimes r is about 10. In some embodiments, R³ is a PEG unit (PEG)ᵣ and r is about 2 to about 10 (e.g., r is about 2 to about 4) or about 18 to about 500.

In some embodiments, s is absent or is about 5 to about 100, and sometimes s is about 5 to about 50 or about 5 to about 25. In certain embodiments, s is about 5 to about 15 and sometimes s is about 10. In some embodiments, s is about 5 or less (e.g., s is 1). In some embodiments, the (R³)ᵣ substituent is linear, and in certain embodiments, the (R³)ᵣ substituent is branched. For linear moieties, s sometimes is less than r (e.g., when R³ is —O—CH₂—CH₂— or —CH₂—CH₂—O—) and at times s is 1. In some embodiments R³ is a linear PEG moiety (e.g., having about 1 to about 1000 PEG units), s is 1 and r is 1. For branched moieties, s sometimes is less than, greater than or equal to r (e.g., when R₃ is —O—CH₂—CH₂— or —CH₂—CH₂—O—), and at times r is 1, s is 1 and p is about 1 to about 1000 (e.g., p is about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In certain embodiments, X² is an amido linking group (e.g., —C(O)NH— or —NH(O)C—); alkyl amido linking group (e.g., —C₁-C₆ alkyl-C(O)NH—, —C₁-C₆ alkyl-NH(O)C—, —C(O)NH—C₁-C₆ alkyl-, —NH(O)C—C₁-C₆ alkyl-, —C₁-C₆ alkyl-NH(O)C—C₁-C₆ alkyl-, —C₁-C₆ alkyl-C(O)NH—C₁-C₆ alkyl-, or —C(O)NH—(CH₂)ₜ—, where t is 1, 2, 3, or 4); substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring) or oxygen-containing moiety (e.g., —O—, —C₁-C₆ alkoxy).

In certain embodiments, one or more substituents terminate the PEG moiety.

In one embodiment, R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In one embodiment, R³ is a PEG moiety terminated with a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In one embodiment, R³ is a PEG moiety terminated with a substituted or unsubstituted aryl or substituted or unsubstituted heteroaryl.

The invention thus provides one or more compounds for use in medical therapy, as well as for the manufacture of a medicament for the treatment of a TLR4 or TLR7 associated condition or symptom. Thus, in one embodiment, the invention provides a method to prevent, inhibit or treat liver disease in a mammal such as a human, bovine, equine, swine, canine, ovine, or feline. The method includes administering to the mammal an effective amount of a composition comprising an amount of a compound having formula (I):

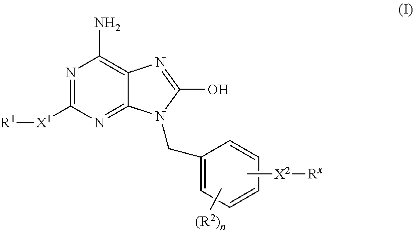

wherein X¹ is —O—, —S—, or —NR^{c}—;

R¹ is hydrogen, (C₁-C₁₀)alkyl, substituted (C₁-C₁₀)alkyl, C₆-C₁₀aryl, or substituted C₆₋₁₀aryl, C₅₋₉heterocyclic, or substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R^{X} is an auxiliary group as described herein;

or a tautomer thereof;

or a pharmaceutically acceptable salt or solvate thereof;

and/or administering a compound of formula (II),

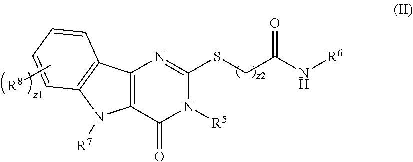

wherein z1 is an integer from 0 to 4, and z2 is an integer from 0 to 5, R⁵ is substituted or unsubstituted cycloalkyl, substituted or unsubstituted heterocycloalkyl, substituted or unsubstituted aryl, or substituted or unsubstituted heteroaryl, R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, R⁷ is hydrogen, or substituted or unsubstituted alkyl, and R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In one embodiment, formula (II) induces compounds that induce type 1 interferon but induce fewer proinflammatory cytokines (see Tables 1, 2 and 3 in Chan et al., J. Med. Chem., 56:4206 (2013)) which tables are incorporated by reference herein.

In some embodiments, a compound of formula (II) is a weak agonist relative to 1Z105:

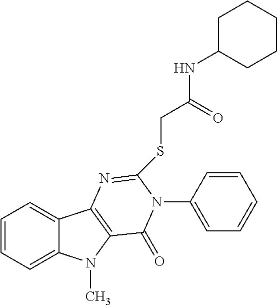 [SMILES: Cn1c2ccccc2c2nc(SCC(=O)NC3CCCCC3)n(-c3ccccc3)c(=O)c21]

### or 1Z65:

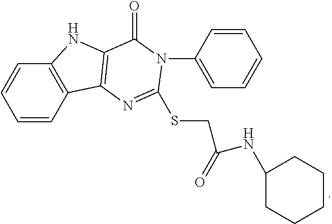 [SMILES: O=C(CSc1nc2c([nH]c3ccccc32)c(=O)n1-c1ccccc1)NC1CCCCC1]

In addition, the invention also provides a pharmaceutical composition comprising at least one TLR4 ligand, a TLR7 ligand, or a combination thereof, or a pharmaceutically acceptable salt, optionally in combination with a pharmaceutically acceptable diluent or carrier.

In one embodiment, the invention provides a prophylactic or therapeutic method for preventing or treating a pathological condition or symptom in a mammal, such as a human, with the compound(s) described herein. Thus, the method includes administering to a mammal in need of such therapy, an effective amount of one or more compounds described herein, or a pharmaceutically acceptable salt thereof. Non-limiting examples of pathological conditions or symptoms that are suitable for treatment include cancers, microbial infections or diseases, e.g., liver, skin or bladder diseases. The invention thus provides compounds for use alone or with other therapeutic agents in medical therapy (e.g., for use as an anti-cancer agent, to prevent, inhibit or treat bacterial diseases, to prevent, inhibit or treat viral diseases, such as hepatitis C and hepatitis B, and generally as agents for enhancing the immune response).

### BRIEF DESCRIPTION OF THE FIGURES

FIG. 1 is a schematic of the development of liver fibrosis/cirrhosis.

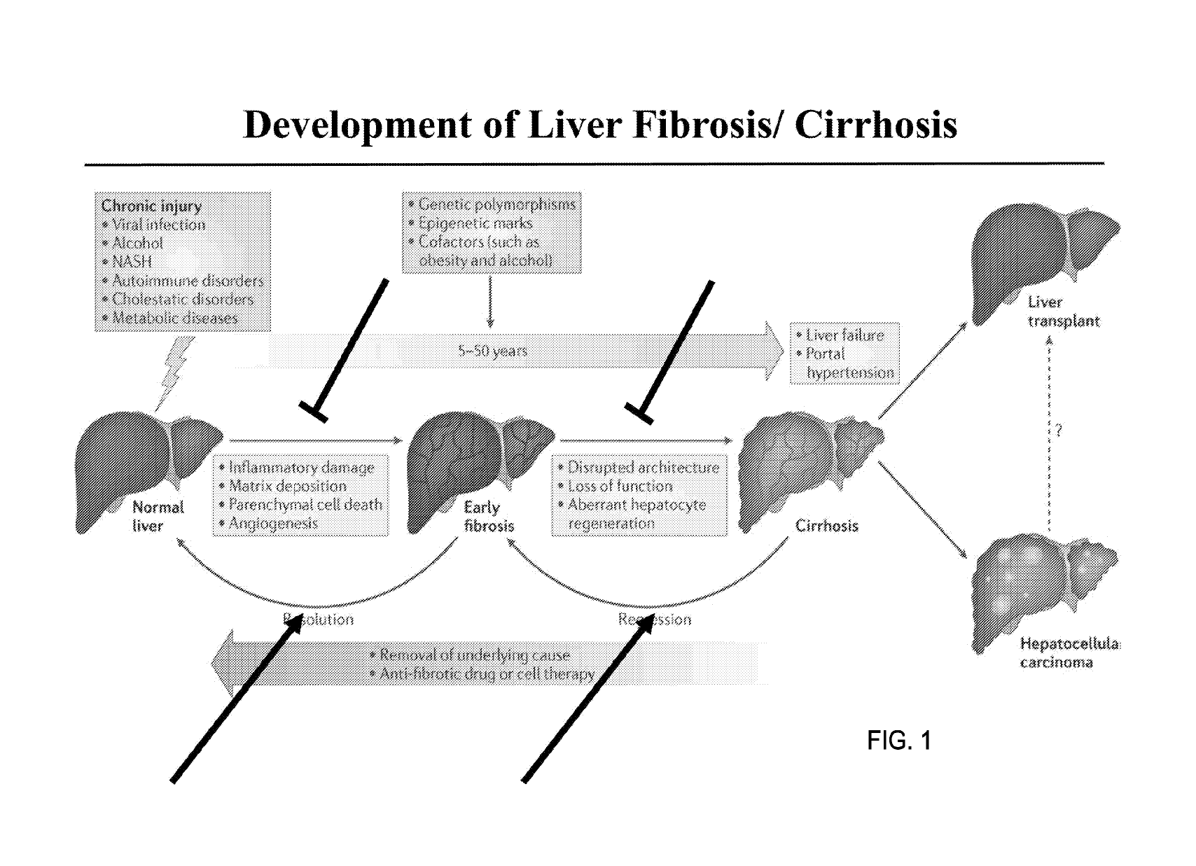

FIG. 2 shows the link between activation of hepatic stellate cells (HSC) and fibrosis. Quiescent HSCs become activated by TGF-3 expressed from Kupffer cells and by TLR4 activation.

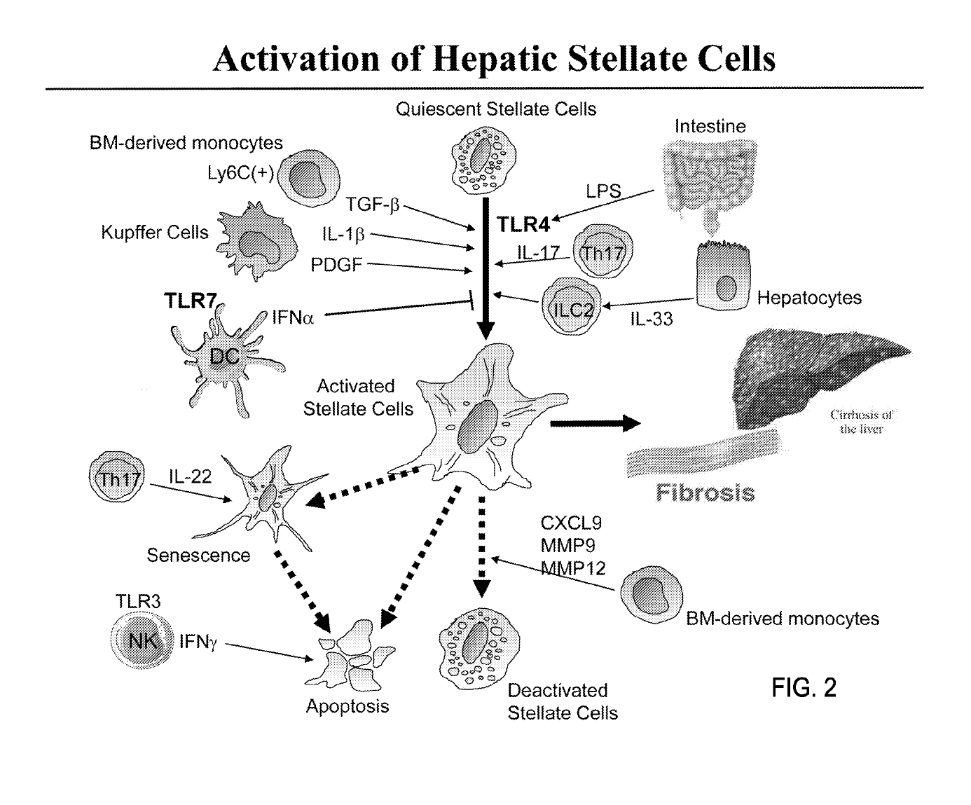

FIG. 3 depicts TLR4 and TLR7 pathways.

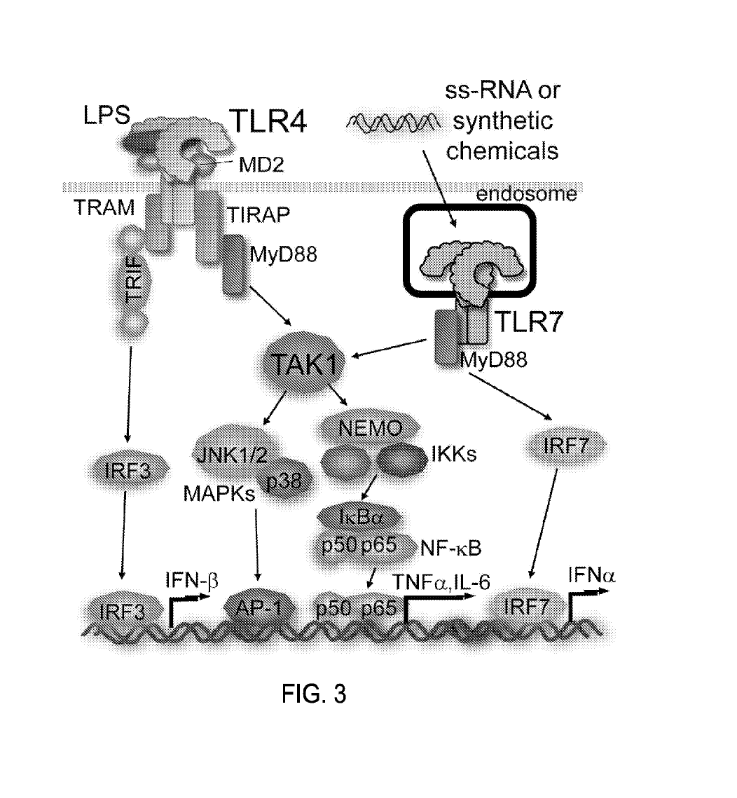

FIG. 4 shows TLR7 signaling is protective against liver fibrosis. Top panels shows Sirius red staining on liver sections of wild type or TLR7 knock out mice and control mice or mice administered R848 (TLR7 agonist) (wt/BDL) bile duct ligation, BDL). Lower panels show % positive staining.

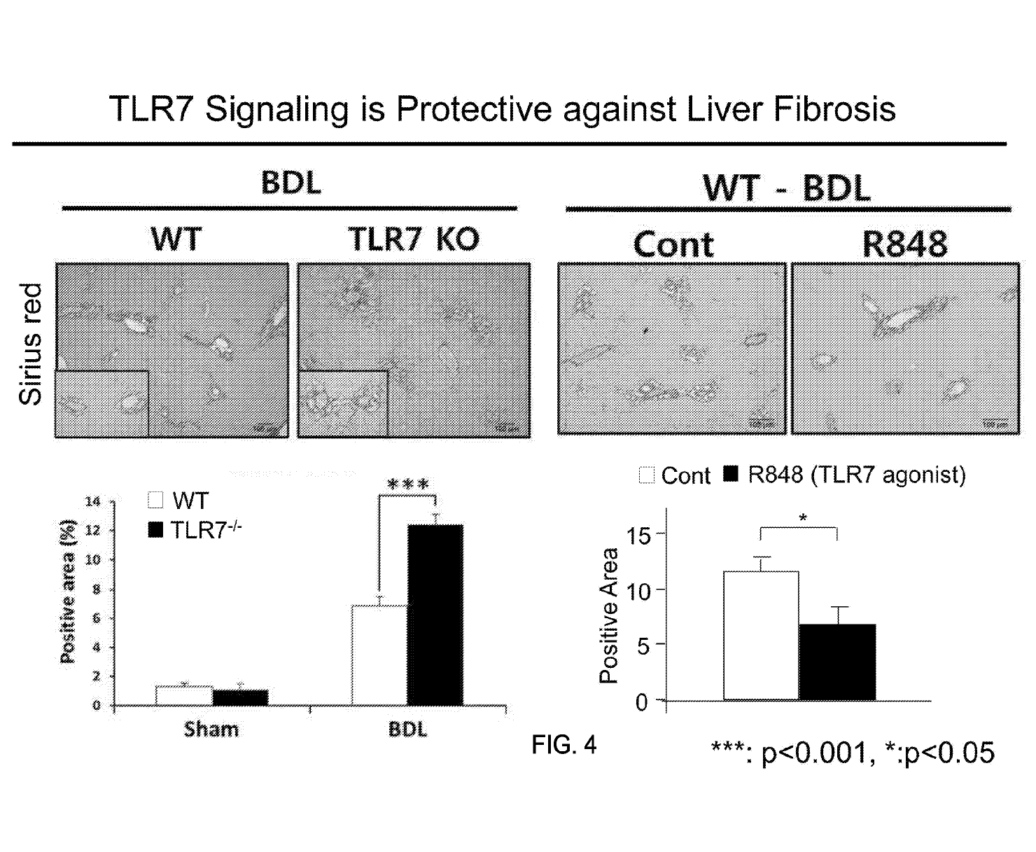

FIG. 5 is a schematic of TLR7 signaling suppressing stellate cell activation.

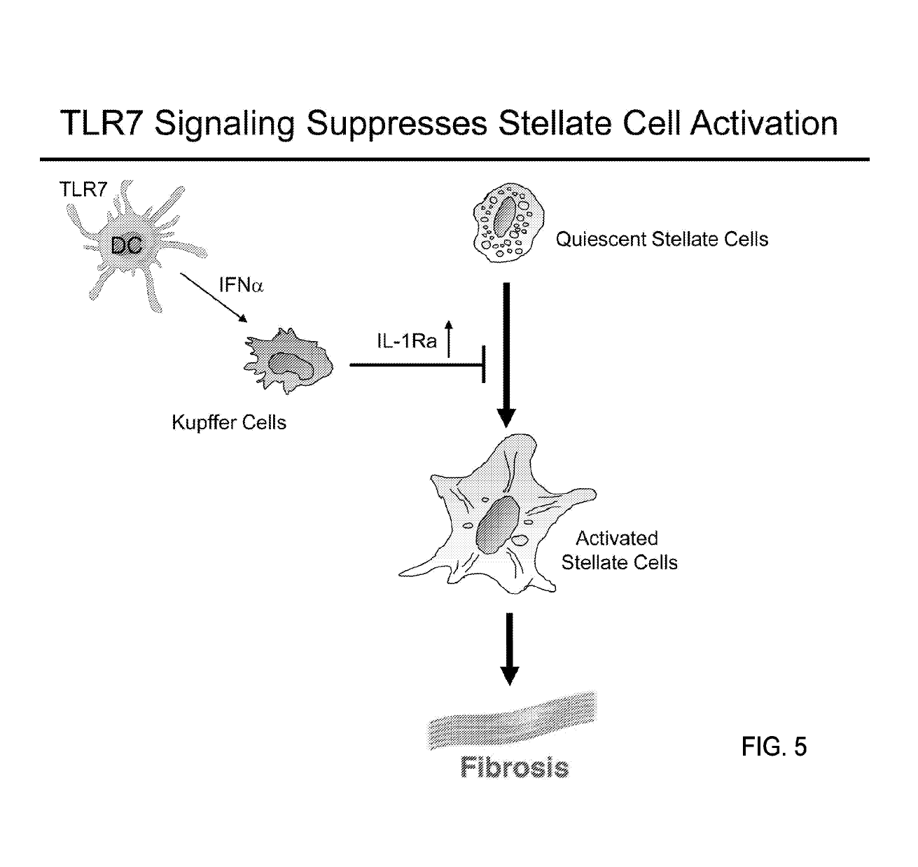

FIG. 6 depicts an exemplary TLR4 ligand (1Z204) as a therapeutic agent to treat liver disease by activating TRIF pathway and by inducing tolerance to subsequent TLR4 stimulation.

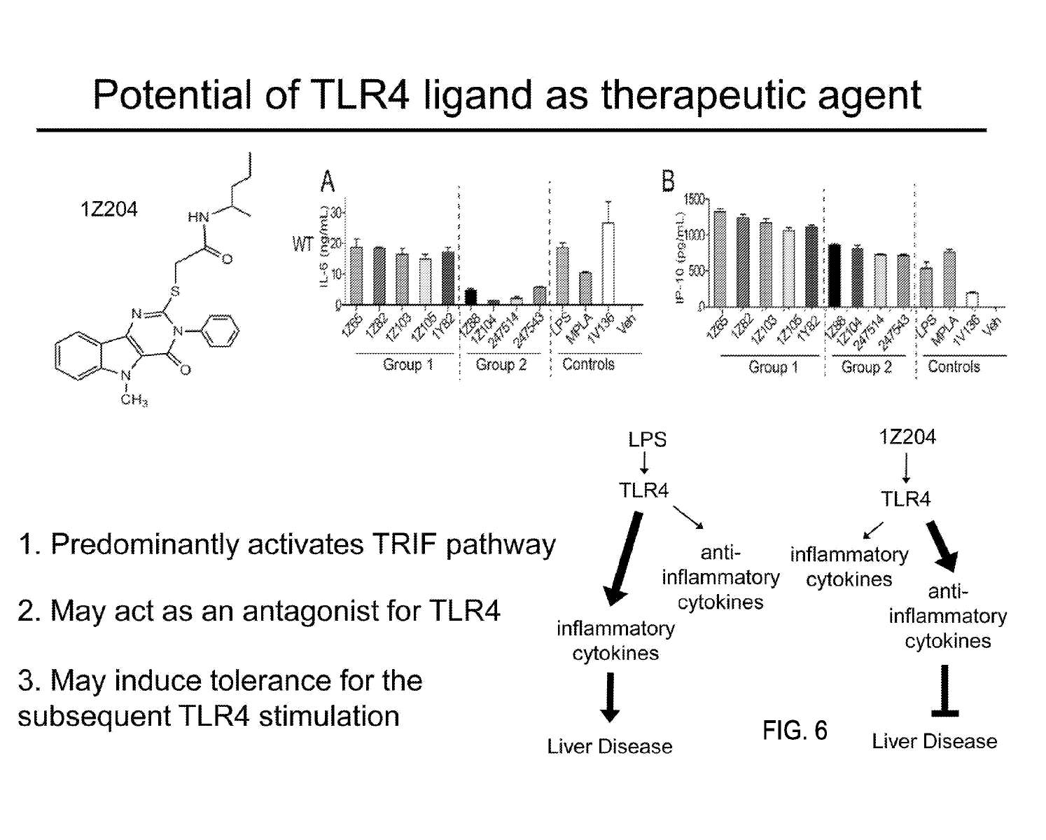

FIG. 7 shows an exemplary TLR7 conjugate (1Z1) and how it may be employed as a therapeutic agent to treat liver disease, e.g., by inducing IFNα, IL10, macrophage death and/or cross-tolerance for other TLRs.

FIG. 8 illustrates progression of alcoholic liver disease.

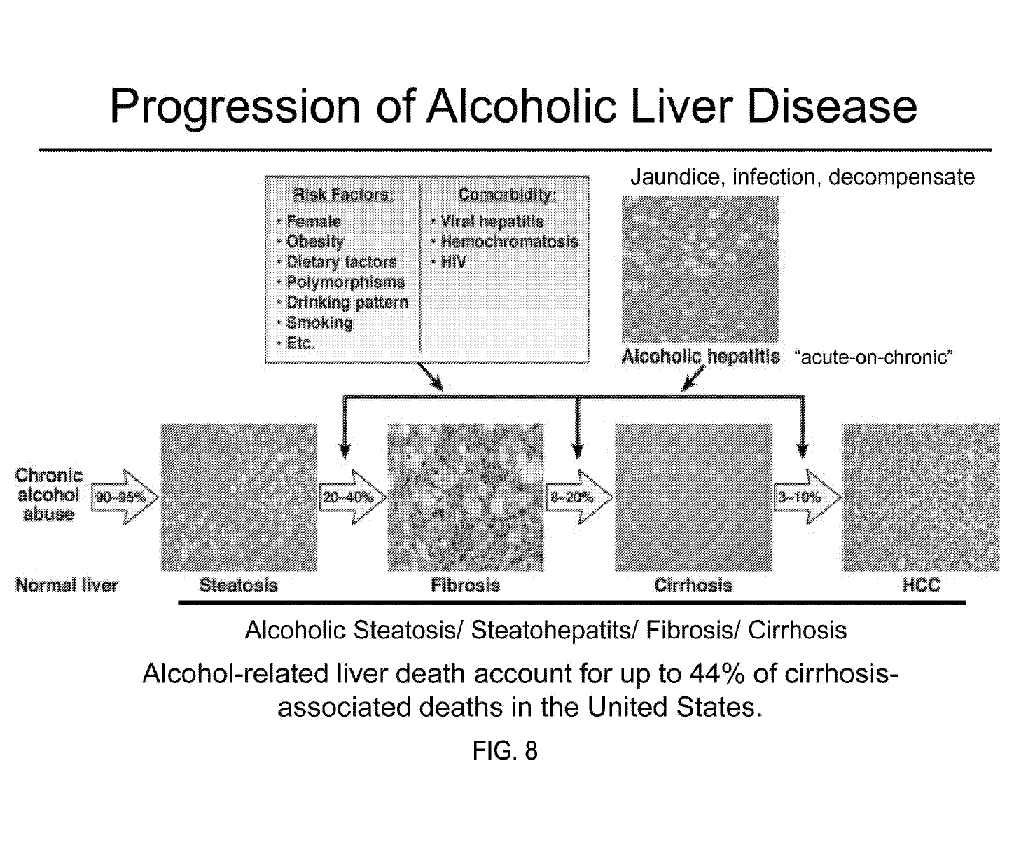

FIG. 9 illustrates pathogenesis of alcoholic liver disease.

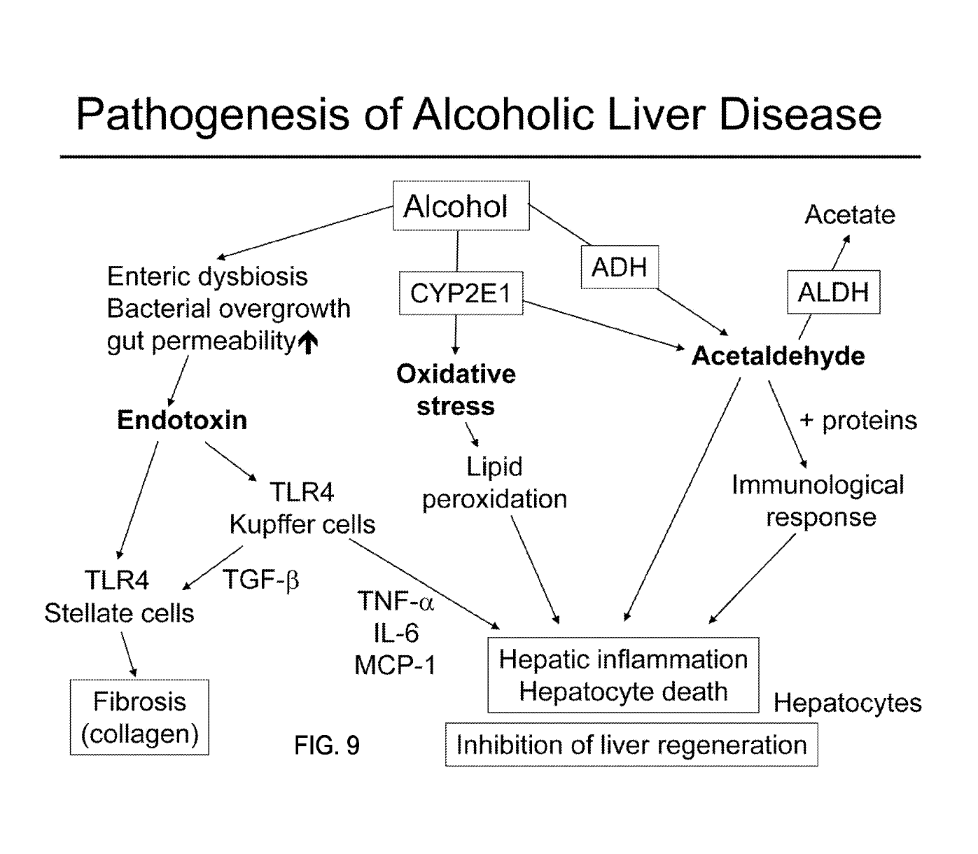

FIG. 10 shows that TLR4 signaling may promote alcoholic steatohepatitis.

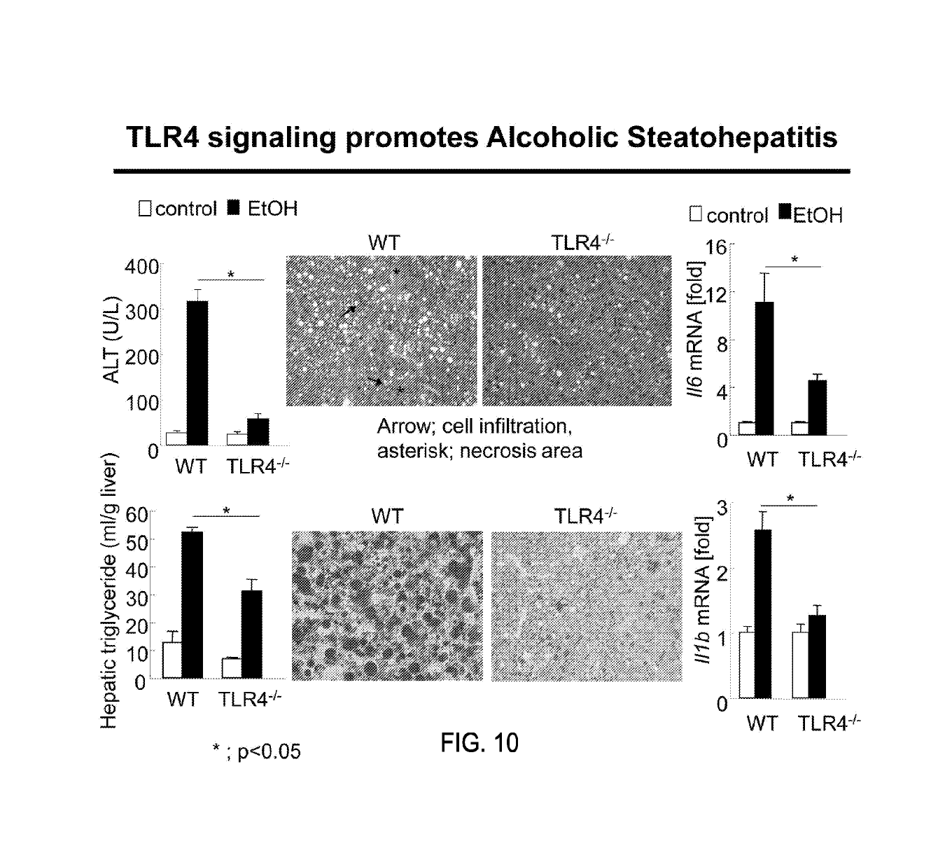

FIG. 11 shows TLR7 deficiency augments alcoholic hepatitis (AH) in a mouse model of AH.

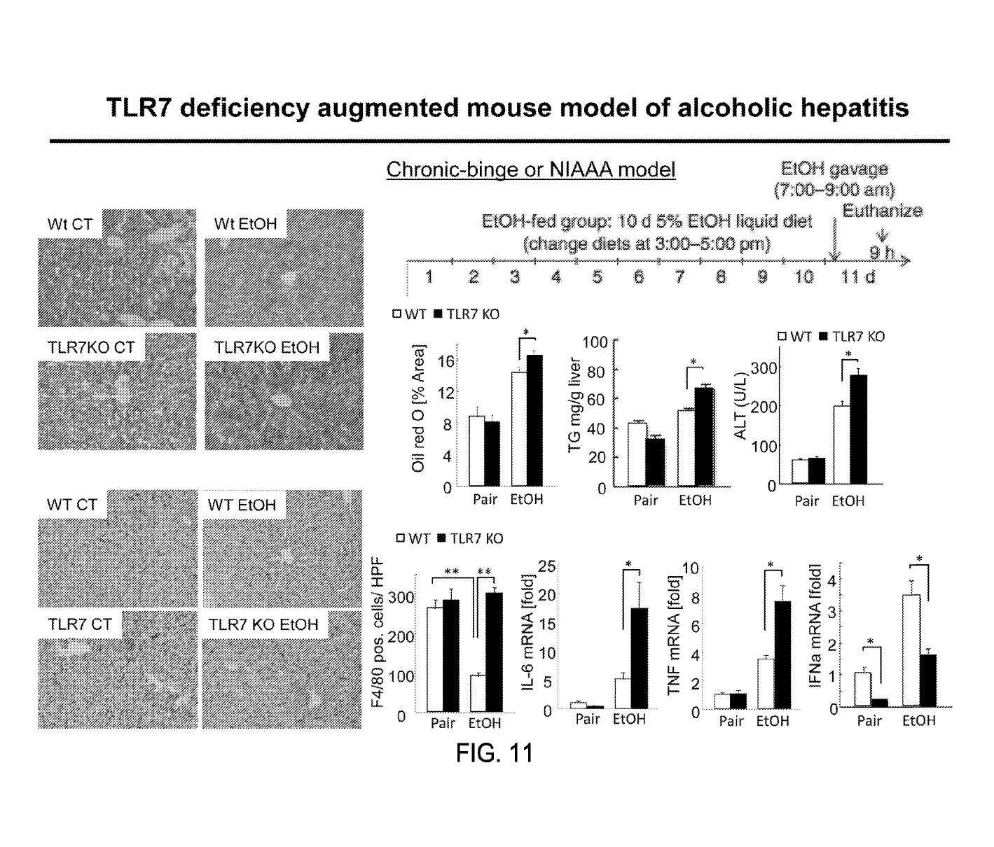

FIG. 12 shows TLR4 and TLR7 ligands suppress AH in a mouse model of alcoholic hepatitis.

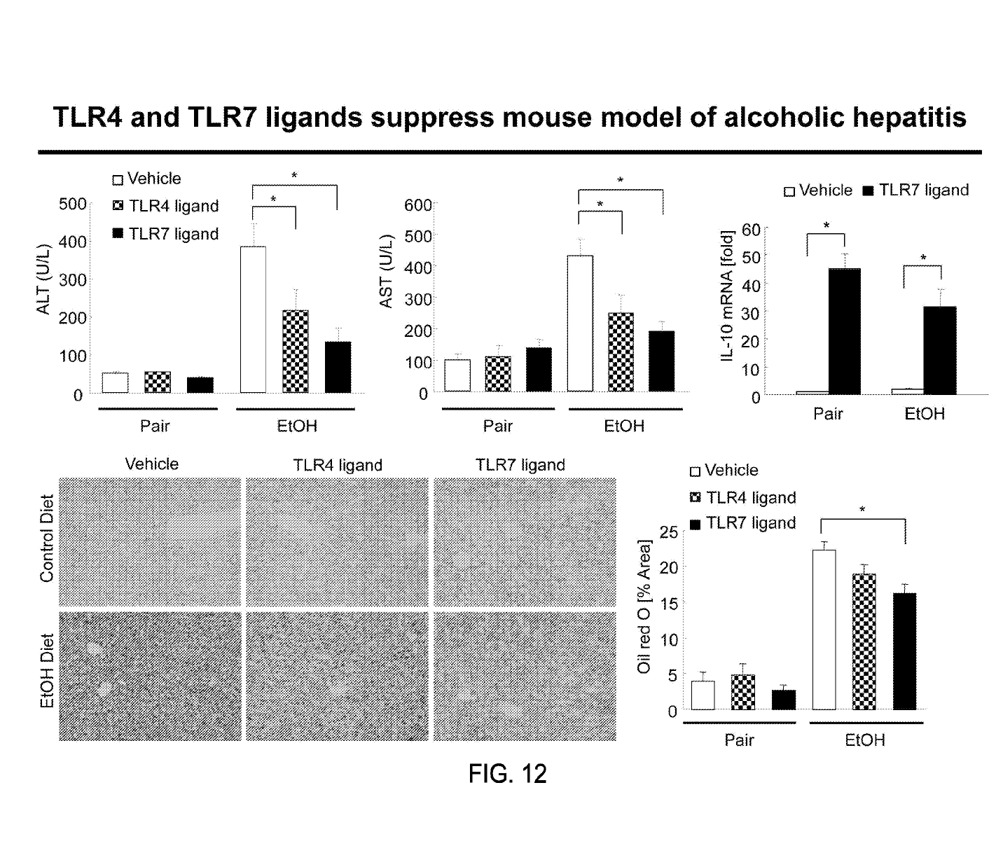

FIG. 13 shows mortality is higher in NASH versus fatty liver alone.

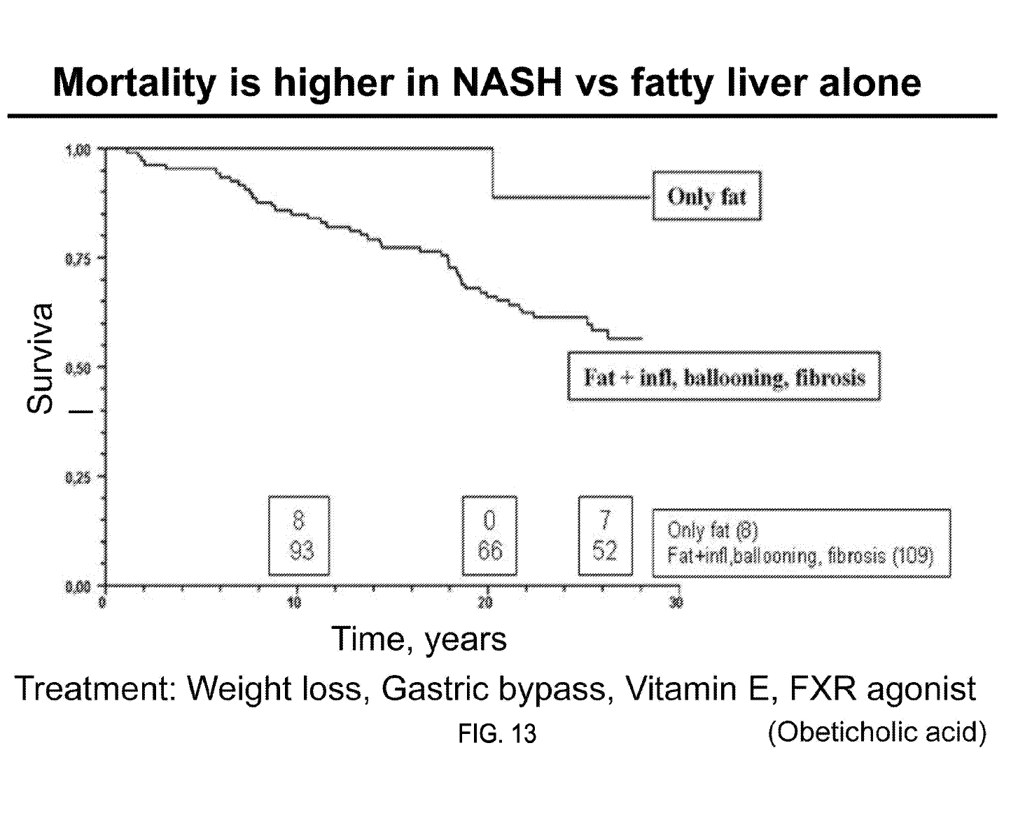

FIG. 14 shows steps in the pathogenesis of NASH.

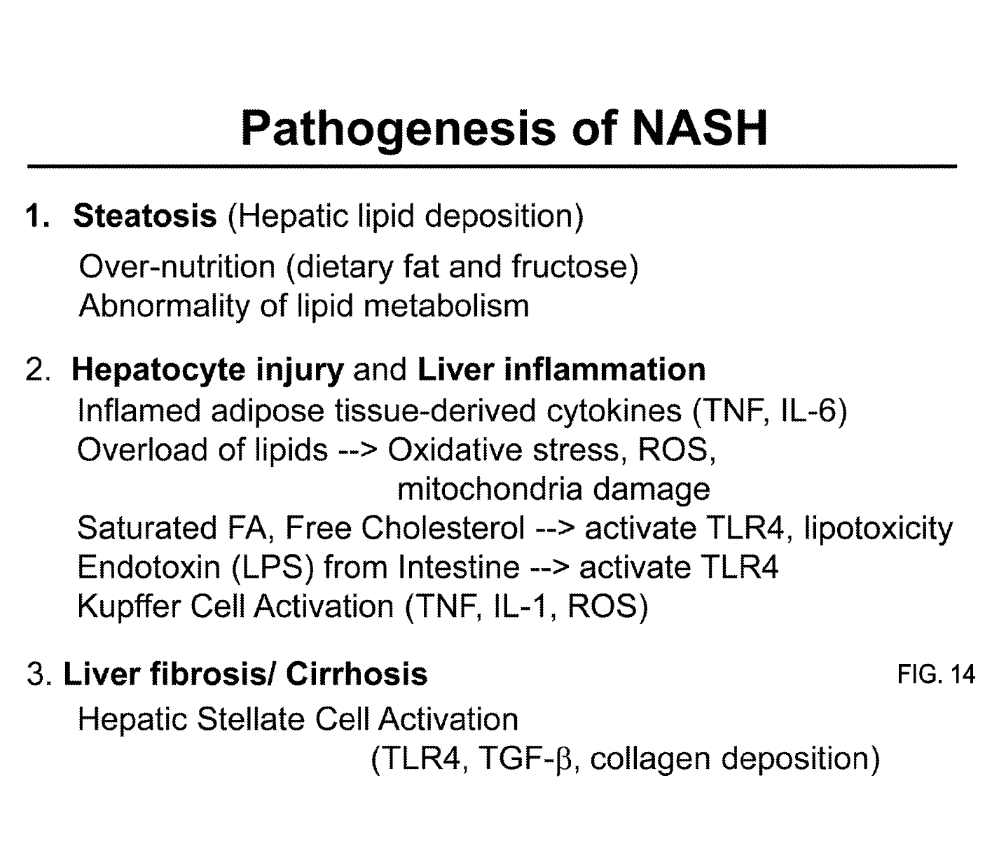

FIG. 15 shows TLR4 signaling promoting NASH development.

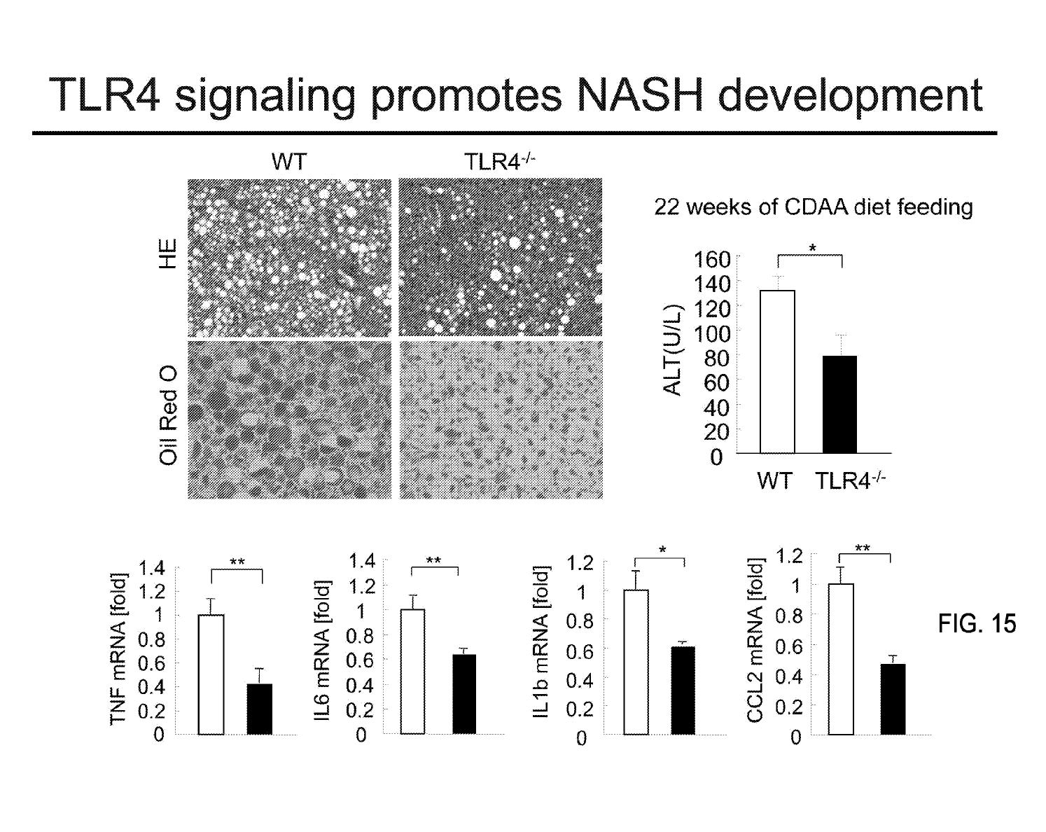

FIG. 16 shows TRIF promoting steatosis but inhibiting inflammation.

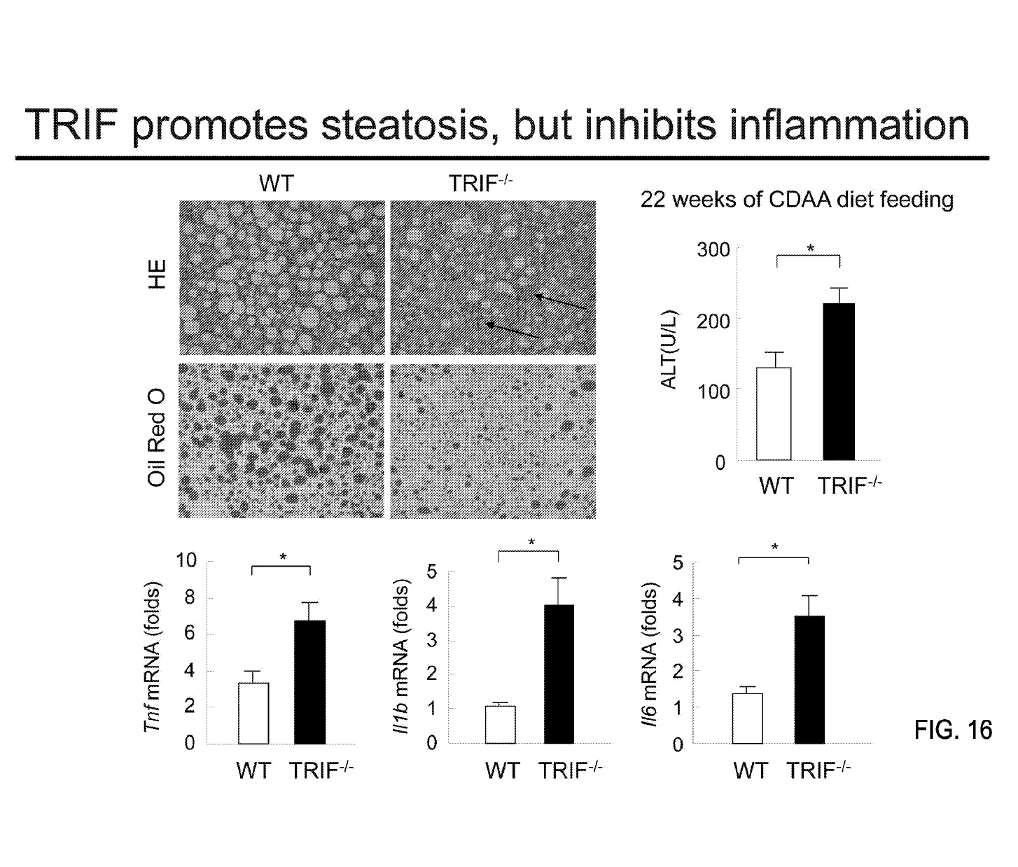

FIG. 17 shows TLR4 and TLR7 ligands suppressing NASH.

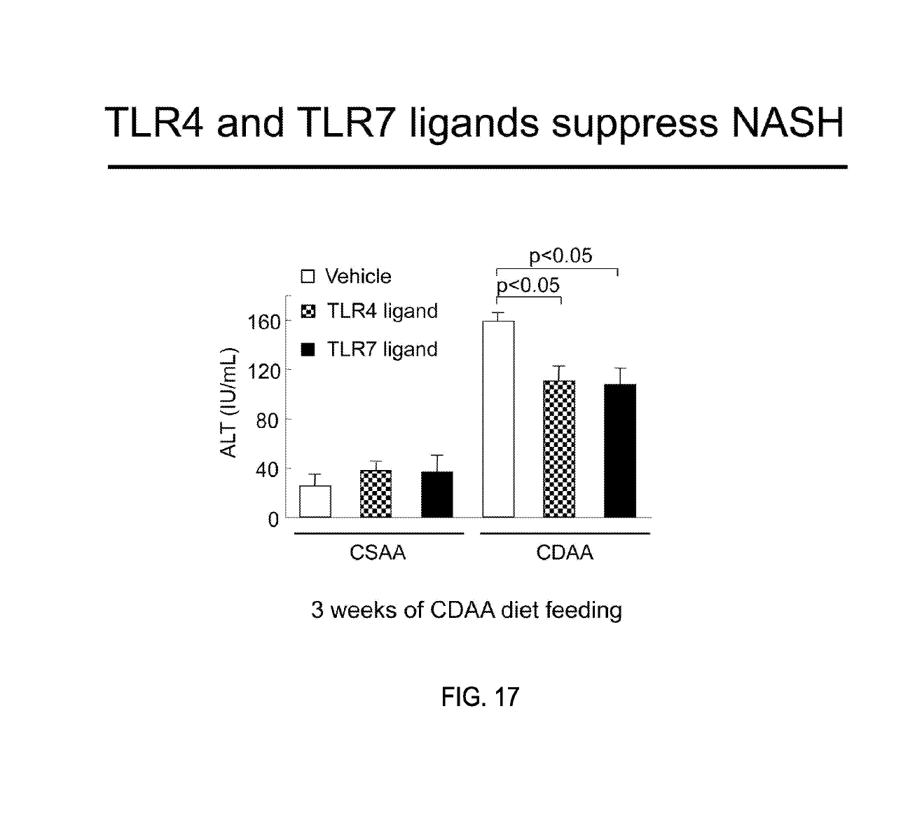

FIG. 18. 1Z1 treatment inhibits CpG-DNA-induced TLR9 signaling-mediated inflammatory response.

FIG. 19. 1Z1 protects against TNFα-mediated hepatocyte death in ethanol-sensitized hepatocytes.

FIG. 20. Therapeutic effects of 1Z204 (a weak TLR4 agonist) on non-alcoholic steatohepatitis mouse model induced by choline-deficient amino acid defined (CDAA) diet.

FIG. 21. Therapeutic effects of 1Z1 (an attenuated TLR7 agonist) in a non-alcoholic steatohepatitis mouse model induced by choline-deficient amino acid defined (CDAA) diet.

### DETAILED DESCRIPTION

### Definitions

A composition is comprised of “substantially all” of a particular compound, or a particular form a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A TLR7 agonist of the invention, or a conjugate thereof, can be prepared as an acid salt or as a base salt, as well as in free acid or free base forms. In solution, certain of the compounds of the invention may exist as zwitterions, wherein counter ions are provided by the solvent molecules themselves, or from other ions dissolved or suspended in the solvent.

The term “toll-like receptor agonist” (TLR agonist) refers to a molecule that binds to a TLR. Synthetic TLR agonists are chemical compounds that are designed to bind to a TLR and activate the receptor.

Within the present invention it is to be understood that a compound of formula (I) or (II) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

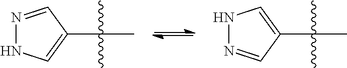 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

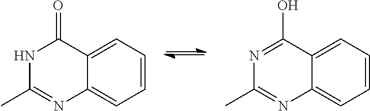

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

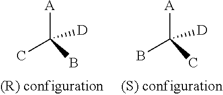

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, Pa., p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof.

The term “TLR” refers to Toll-like receptors which are are components of the innate immune system that regulate NF_{K}B activation.

The terms “TLR modulator,” “TLR immunomodulator” and the like as used herein refer, in the usual and customary sense, to compounds which agonize or antagonize a Toll Like Receptor. See e.g., PCT/US2010/000369, Hennessy, E. J., et al., Nature Reviews 2010, 9:283-307; PCT/US2008/001631; PCT/US2006/032371; PCT/US2011/000757. Accordingly, a “TLR agonist” is a TLR modulator which agonizes a TLR, and a “TLR antagonist” is a TLR modulator which antagonizes a TLR.

The term “TLR4” as used herein refers to the product of the TLR4 gene, and homologs, isoforms, and functional fragments thereof: Isoform 1 (NCBI Accession NP_612564.1); Isoform 2 (NCBI Accession NP_003257.1); Isoform 3 (NCBI Accession NP_612567.1).

The term “TLR7” as used herein refers to the product (NCBI Accession AAZ99026) of the TLR7 gene, and homologs, and functional fragments thereof.

The term “TLR8” as used herein refers to the product (NCBI Accession AAZ95441) of the TLR8 gene, and homologs, and functional fragments thereof.

### TLR4, TLR7, TLR8 and TLR9

Toll-like receptors (TLRs) are pattern recognition receptors that recognize conserved microbial products, known as pathogen-associated molecular patterns (PAMPs). TLR4 recognizes LPS. TLR4 signaling activates MyD88 and TRIF-dependent pathways. MyD88 pathway activates NF-κB and JNK to induce inflammatory response. TRIF pathway activates IRF3 to induce IFN-α production.

TLR4 is expressed predominately on monocytes, mature macrophages and dendritic cells, mast cells and the intestinal epithelium. TLR modulators (antagonists) for TLR4 include NI-0101 (Hennessy 2010, Id.), 1A6 (Ungaro, R., et al., Am. J. Physiol. Gastrointest. Liver Physiol. 2009, 296:G1167-G1179), AV411 (Ledeboer, A., et al., Neuron Glia Biol. 2006, 2:279-291; Ledeboer, A., et al., Expert Opin. Investig. Drugs 2007, 16:935-950), Eritoran (Mullarkey, M., et al., J. Pharmacol. Exp. Ther. 2003, 305:1093-1102), and TAK-242 (Li, M., et al., Mol. Pharmacol. 2006, 69:1288-1295). TLR modulators (agonists) for TLR4 include Pollinex® Quattro (Baldrick, P., et al., J. Appl. Toxicol. 2007, 27:399-409; DuBuske, L., et al., J. Allergy Clin. Immunol. 2009, 123:S216). TLR7 signaling activates MyD88-dependent pathway and IRF7-dependent signaling. IRF7 pathway induces IFN-α production.

TLR7 senses ss-RNA or synthetic chemicals (Imiquimod, R848). TLR7 and TLR8 are found in endosomes of monocytes and macrophages, with TLR7 also being expressed on plasmacytoid dendritic cells, and TLR8 also being expressed in mast cells. Both these receptors recognize single stranded RNA from viruses. Synthetic ligands, such as R-848 and imiquimod, can be used to activate the TLR7 and TLR8 signaling pathways. See e.g., Caron, G., et al., J. Immunol. 2005, 175:1551-1557. TLR9 is expressed in endosomes of monocytes, macrophages and plasmacytoid dendritic cells, and acts as a receptor for unmethylated CpG islands found in bacterial and viral DNA. Synthetic oligonucleotides that contain unmethylated CpG motifs are used to activate TLR9. For example, class A oligonucleotides target plasmacytoid dendritic cells and strongly induce IFNa production and antigen presenting cell maturation, while indirectly activating natural killer cells. Class B oligonucleotides target B cells and natural killer cells and induce little interferon-a (IFNa). Class C oligonucleotides target plasmacytoid dendritic cells and are potent inducers of IFNa. This class of oligonucleotides is involved in the activation and maturation of antigen presenting cells, indirectly activates natural killer cells and directly stimulates B cells. See e.g., Vollmer, J., et al., Eur. J. Immunol. 2004, 34:251-262; Strandskog, G., et al., Dev. Comp. Immunol. 2007, 31:39-51.

Reported TLR modulators (agonist) for TLR7 include ANA772 (Kronenberg, B. & Zeuzem, S., Ann. Hepatol. 2009, 8:103-112), Imiquimod (Somani, N. & Rivers, J. K., Skin Therapy Lett. 2005, 10:1-6), and AZD8848 (Hennessey 2010, Id.) TLR modulators (agonist) for TLR8 include VTX-1463 (Hennessey 2010, Id.) TLR modulators (agonist) for TLR7 and TLR8 include Resiquimod (Mark, K. E., et al., J. Infect. Dis. 2007, 195:1324-1331; Pockros, P. J., et al., J. Hepatol. 2007, 47:174-182). TLR modulators (antagonists) for TLR7 and TLR9 include IRS-954 (Barrat, F. J., et al., Eur. J. Immunol. 2007, 37:3582-3586), and IMO-3100 (Jiang, W., et al., J. Immunol. 2009, 182:48.25). TLR9 agonists include SD-101 (Barry, M. & Cooper, C., Expert Opin. Biol. Ther. 2007, 7:1731-1737), IMO-2125 (Agrawal, S. & Kandimalla, E. R., Biochem. Soc. Trans. 2007, 35:1461-1467), Bio Thrax plus CpG-7909 (Gu, M., et al., Vaccine 2007, 25:526-534), AVE0675 (Parkinson, T., Curr. Opin. Mol. Ther. 2008, 10:21-31), QAX-935 (Panter, G., et al., Curr. Opin. Mol Ther. 2009, 11:133-145), SAR-21609 (Parkinson 2008, Id.), and DIMS0150 (Pastorelli, L., et al., Expert Opin. Emerg. Drugs 2009, 14:505-521).

### TLR7 Ligands and Conjugates Thereof

With regard to TLR7 ligands and conjugates thereof, as used herein, the terms “alkyl,” “alkenyl” and “alkynyl” may include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2 propenyl, 3 butynyl, and the like. The total number of carbon atoms in each such group is sometimes described herein, e.g., when the group can contain up to ten carbon atoms it can be represented as 1-10C or as C₁-C₁₀ or C₁₋₁₀. When heteroatoms (N, O and S typically) are allowed to replace carbon atoms as in heteroalkyl groups, for example, the numbers describing the group, though still written as e.g. C₁-C₆, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain one 10C (alkyl) or two 10C (alkenyl or alkynyl). For example, they contain one 8C (alkyl) or two 8C (alkenyl or alkynyl). Sometimes they contain one 4C (alkyl) or two 4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C₁-C₈ alkyl, C₂-C₈ heteroalkyl, C₁-C₈ acyl, C₂-C₈ heteroacyl, C₆-C₁₀ aryl or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group.

“Acetylene” substituents may include 2-10C alkynyl groups that are optionally substituted, and are of the formula —C≡C-Ri, wherein Ri is H or C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₁-C₈ acyl, C₂-C₈ heteroacyl, C₆-C₁₀ aryl, C₅-C₁₀ heteroaryl, C₇-C₁₂ arylalkyl, or C₆-C₁₂ heteroarylalkyl, and each Ri group is optionally substituted with one or more substituents selected from halo, ═O, ═N—CN, ═N—OR′, ═NR′, OR′, NR′2, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C₁-C₆ alkyl, C₂-C₆ heteroalkyl, C₁-C₆ acyl, C₂-C₆ heteroacyl, C₆-C₁₀ aryl, C₅-C₁₀ heteroaryl, C₇₋₁₂ arylalkyl, or C₆₋₁₂ heteroarylalkyl, each of which is optionally substituted with one or more groups selected from halo, C₁-C₄ alkyl, C₁-C₄ heteroalkyl, C₁-C₆ acyl, C₁-C₆ heteroacyl, hydroxy, amino, and ═O; and wherein two R′ can be linked to form a 3-7 membered ring optionally containing up to three heteroatoms selected from N, O and S. In some embodiments, Ri of —C≡C—Ri is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain one to three O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5 membered rings as well as 6 membered rings. Typical heteroaromatic systems include monocyclic C₅-C₆ aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. For example, the monocyclic heteroaryls may contain 5-6 ring members, and the bicyclic heteroaryls contain 8-10 ring members.

Aryl and heteroaryl moieties may be substituted with a variety of substituents including C₁-C₈ alkyl, C₂-C₈ alkenyl, C₂-C₈ alkynyl, C₅-C₁₂ aryl, C₁-C₈ acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₆-C₁₀ aryl, C₅-C₁₀ heteroaryl, C₇-C₁₂ arylalkyl, or C₆-C₁₂ heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. For example, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group may include a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C₇-arylalkyl group, and phenylethyl is a C₈-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and for instance n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R² is alkyl, this alkyl may optionally be substituted by the remaining substituents listed as embodiments for R² where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

In various embodiments, the invention provides a method to prevent, inhibit or treat liver disease such as one associated with inflammation in a mammal. The methods include administering to a mammal in need thereof an effective amount of a compound of Formula (I):

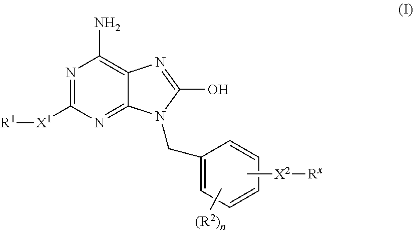

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₆)cycloalkyl, substituted (C₃-C₆)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R^{x} is an auxiliary group, for example, —(R³)ᵣ—(R⁴)ₛ)ₚ wherein each R³ independently is a polyethylene glycol (PEG) moiety; wherein each R⁴ independently is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic; wherein r is 1 to 1000, where s is 1 to 100 and where p is 1 to 100;

or a tautomer thereof;

or a pharmaceutically acceptable salt or solvate thereof.

In one embodiment, R³ is a PEG moiety.

In some embodiments, a PEG reactant has a structure CH₃O(CH₂CH₂O)ₙ—X—NHS*, where X can be —COCH₂CH₂COO—, —COCH₂CH₂CH₂ COO—, —CH₂COO—, and —(CH₂)₅COO—. In certain embodiments, a PEG reactant has a structure

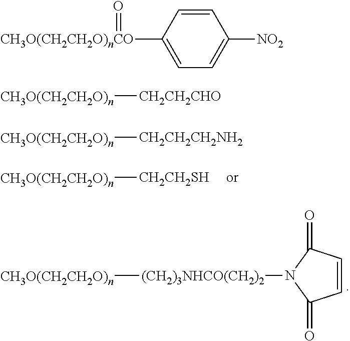

Certain PEG reactants are bifunctional in some embodiments. Examples of bifunctional PEG reactants have a structure X—(OCH₂CH₂)n-X, where X is (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), Aminopropyl (—CH₂CH₂CH₂NH₂) or 2-Sulfanylethyl (—CH₂CH₂SH) in some embodiments.

In certain embodiments, some PEG reactants are heterofunctional. Examples of heterofunctional PEG reactants have the structures

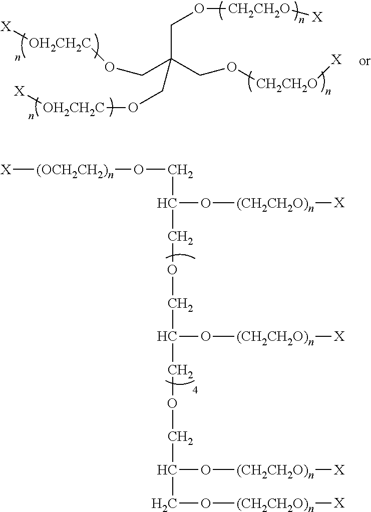

where X can be (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), 3-aminopropyl (—CH₂CH₂CH₂NH₂), 2-Sulfanylethyl (—CH₂CH₂SH), 5-(N-Succinimidyloxycarbonyl)pentyl (—(CH₂)₅COO—NHS], or p-Nitrophenyloxycarbonyl, (—CO₂-p-C₆H₄NO₂), in some embodiments.

Certain branched PEG reactants also may be utilized, such as those having a structure:

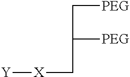

where X is a spacer and Y is a functional group, including, but not limited to, maleimide, amine, glutaryl-NHS, carbonate-NHS or carbonate-p-nitrophenol, in some embodiments. An advantage of branched chain PEG reactants is that they can yield conjugation products that have sustained release properties.

A PEG reactant also may be a heterofunctional reactant, such as

HO(CH₂CH₂O)ₙ—CH₂CH₂CH₂NH₂

HCl.H₂N—CH₂CH₂CH₂O(CH₂CH₂O)ₙ—(CH₂)₅COOH and

HO(CH₂CH₂O)ₙ—CH₂CH₂CHO

in certain embodiments. In some embodiments, Boc*-protected-Amino-PEG-Carboxyl-NHS or Maleimide-PEG-Carboxyl-NHS reactants can be utilized.

In certain embodiments, a comb-shaped polymer may be utilized as a PEG reactant to incorporate a number of PEG units into a conjugate. An example of a comb-shaped polymer is shown hereafter.

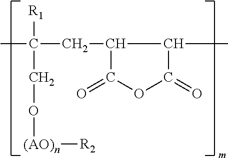

A PEG reactant, and/or a PEG conjugate product, can in some embodiments have a molecular weight ranging between about 5 grams per mole to about 100,000 grams per mole. In some embodiments, a PEG reactant, and/or a PEG conjugate product, has a average, mean or nominal molecular weight of about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000 or 90000 grams per mole. In some embodiments the PEG moiety in a compound herein is homogeneous and the molecule weight of the PEG moiety is the same for each molecule of a particular batch of compound (e.g., R³ is one PEG unit and r is 2 to 10).

In various embodiments, X² in formula (I) can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

Certain non-limiting examples of X² in formula (I) include —(Y)_{y}—, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C₁₋₆ alkoxy C₁₋₆ alkenyl, amino, cyano, halogen or aryl. In certain embodiments, a linker sometimes is a —C(Y′)(Z′)—C(Y″)(Z″)— linker, where each Y′, Y″, Z′ and Z″ independently is hydrogen C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C1-6 alkoxy C₁₋₆ alkenyl, amino, cyano, halogen or aryl.

Another specific value for X² in formula (I) is

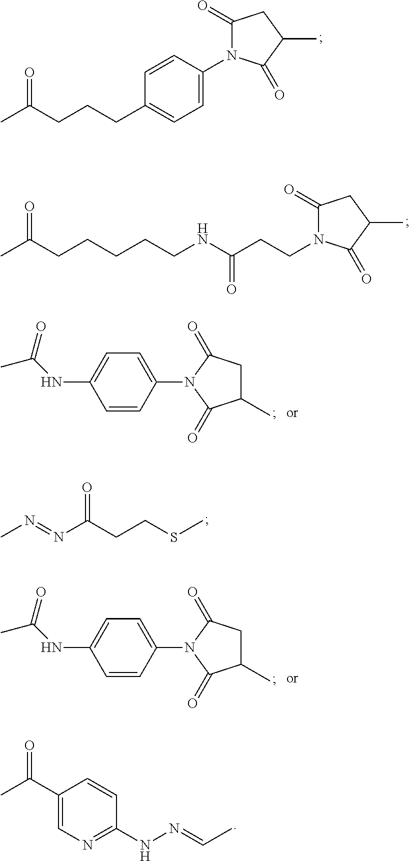

Another specific value for X² is

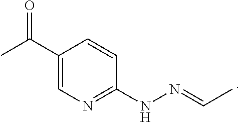

In various embodiments, X² can be C(O), or can be any of

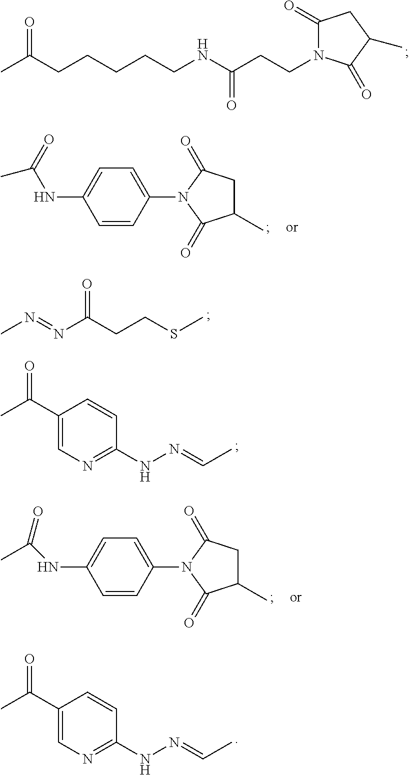

In various embodiments, X¹ in formula (I) can be oxygen.

In various embodiments, X¹ in formula (I) can be sulfur, or can be —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl. More specifically, X¹ can be —NH—.

In various embodiments, R¹ and R^{c} in formula (I) taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ in formula (I) can be a C₁-C₁₀ alkyl substituted with C₁₋₆ alkoxy.

In various embodiments, R¹ in formula (I) can be hydrogen, C₁₋₄alkyl, or substituted C₁₋₄alkyl. More specifically, R¹ can be hydrogen, methyl, ethyl, propyl, butyl, hydroxyC₁₋₄alkylene, or C₁₋₄alkoxyC₁₋₄alkylene. Even more specifically, R¹ can be hydrogen, methyl, ethyl, methoxyethyl, or ethoxyethyl.

In various embodiments, R² in formula (I) can be absent, or R² can be halogen or C₁₋₄alkyl. More specifically, R² can be chloro, bromo, methyl, or ethyl.

In one embodiment, R^{x} in formula (I) is ((R³)ᵣ—(R⁴)ₛ)ₚ or is R³. In one embodiment, R³ is a PEG moiety or a derivative of a PEG moiety. In certain embodiment R³ is —O—CH₂—CH₂— or —CH₂—CH₂—O—. In one embodiment, a PEG moiety can include one or more PEG units. A PEG moiety can include about 1 to about 1,000 PEG units, including, without limitation, about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800 or 900 units, in some embodiments. In certain embodiments, a PEG moiety can contain about 1 to 5 up to about 25 PEG units, about 1 to 5 up to about 10 PEG units, about 10 up to about 50 PEG units, about 18 up to about 50 PEG units, about 47 up to about 150 PEG units, about 114 up to about 350 PEG units, about 271 up to about 550 PEG units, about 472 up to about 950 PEG units, about 50 up to about 150 PEG units, about 120 up to about 350 PEG units, about 250 up to about 550 PEG units or about 650 up to about 950 PEG units. A PEG unit is —O—CH₂—CH₂— or —CH₂—CH₂—O— in certain embodiments. In some embodiments, R⁴ is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic.

In some embodiments, r is about 5 to about 100, and sometimes r is about 5 to about 50 or about 5 to about 25. In certain embodiments, r is about 5 to about 15 and sometimes r is about 10. In some embodiments, R³ is a PEG unit (PEG)ᵣ and r is about 2 to about 10 (e.g., r is about 2 to about 4) or about 18 to about 500.

In some embodiments, s is about 5 to about 100, and sometimes s is about 5 to about 50 or about 5 to about 25. In certain embodiments, s is about 5 to about 15 and sometimes s is about 10. In some embodiments, s is about 5 or less (e.g., s is 1). In some embodiments, the (R³)ᵣ substituent is linear, and in certain embodiments, the (R³)ᵣ substituent is branched. For linear moieties, s sometimes is less than r (e.g., when R³ is —O—CH₂—CH₂— or —CH₂—CH₂—O—) and at times s is 1. In some embodiments R³ is a linear PEG moiety (e.g., having about 1 to about 1000 PEG units), s is 1 and r is 1. For branched moieties, s sometimes is less than, greater than or equal to r (e.g., when R³ is —O—CH₂—CH₂— or —CH₂—CH₂—O—), and at times r is 1, s is 1 and p is about 1 to about 1000 (e.g., p is about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In some embodiments R³ is —O—CH₂—CH₂— or —CH₂—CH₂—O— and r is about 1 to about 1000 (e.g., about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800, 900 or 1000).

In certain embodiments, X² is an amido linking group (e.g., —C(O)NH— or —NH(O)C—); alkyl amido linking group (e.g., —C₁-C₆ alkyl-C(O)NH—, —C₁-C₆ alkyl-NH(O)C—, —C(O)NH—C₁-C₆ alkyl-, —NH(O)C—C₁-C₆ alkyl-, —C₁-C₆ alkyl-NH(O)C—C₁-C₆ alkyl-, —C₁-C₆ alkyl-C(O)NH—C₁-C₆ alkyl-, or —C(O)NH—(CH₂)ₜ—, where t is 1, 2, 3, or 4); substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring) or oxygen-containing moiety (e.g., —O—, —C₁-C₆ alkoxy).

In various embodiments, the mammal can be a human.

In various embodiments, the composition can be intranasally administered, or can be dermally administered, or can be systemically administered.

In various embodiments, a conjugate can be can be incorporated into a nanoparticle such as those described in WO 2010/083337, the disclosure of which is incorporated by reference herein.

### TLR4 Ligands

As used herein with regard to TLR4 ligands, the term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, npropyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. In one embodiment those groups have 10 or fewer carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples ofheterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to 15 multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quatemized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =0, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR —C(NR′R″R″′)═NR″″, —NR—C(NR′R″)═NR″′, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R″′, and R″″ in one embodiment each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —CN, —NO₂, —R′, —N₃, —CH(Ph)z, fluoro(C₁-C₄)alkoxy, and fluoro(C₁-C₄)alkyl, in a number ranging from zero to the total number of open valences on the aromatic ring system; and where R′, R″, R″′, and R″″ are in one embodiment independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″′)_{d}—, where sand dare independently integers of from 0 to 3, and X′ is —O—, —NR′—, —S—, —S(O)—, —S(O)₂—, or —S(O)₂NR′—. The substituents R, R′, R″, and R″′ are in one embodiment independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —CCl₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In some embodiments, a compound as described herein may include multiple instances of a substituent, e.g., R⁵, R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C}. In such embodiments, each substituent may optional be different at each occurrence and be appropriately labeled to distinguish each group for greater clarity. For example, where each R^{5A} is different, they may be referred to as e.g., R^{5A.1}, R^{5A.2}, R^{5A.3}, R^{5A.4}, R^{5A.5}. Similarly, where any of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C} multiply occur, the definition of each occurrence of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C} assumes the definition of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C}, respectively.

In one aspect, there is provided a compound having formula (II):

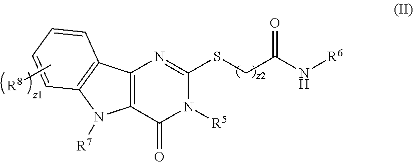

or a pharmaceutically acceptable salt thereof. In formula (II), z1 is an integer from 0 to 4, and z2 is an integer from 0 to 5, R⁵ is substituted or unsubstituted cycloalkyl, substituted or unsubstituted heterocycloalkyl, substituted or unsubstituted aryl, or substituted or unsubstituted heteroaryl, R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, R⁷ is hydrogen, or substituted or unsubstituted alkyl, and R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In one embodiment, R⁵ is R^{5A}-substituted or unsubstituted cycloalkyl, R^{5A} substituted or unsubstituted heterocycloalkyl, R^{5A} substituted or unsubstituted aryl, or R^{5A} substituted or unsubstituted heteroaryl. R^{5A} is independently halogen, —CN, —CF₃, —CCl₃, —OH, —NH₂, —SO₂, —COOH, oxo, nitro, —SH, —CONH₂, R^{5B}-substituted or unsubstituted alkyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl. R^{5B} is independently halogen, —CN, —CF₃, —CCl₃, —OH, —NH₂, —SO₂, —COOH, oxo, nitro, —SH, —CONH₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl. R^{5C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁶ is R^{6A}-substituted or unsubstituted alkyl, R^{6A} substituted or unsubstituted heteroalkyl, R^{6A} substituted or unsubstituted cycloalkyl, R^{6A} substituted or unsubstituted heterocycloalkyl, R^{6A} substituted or unsubstituted aryl, or R^{6A} substituted or unsubstituted heteroaryl. R^{6A} is independently halogen, —CN, —CF₃, —CCl₃, —OH, —NH₂, —SO₂, —COOH, oxo, nitro, —SH, —CONH₂, R^{6B}-substituted or unsubstituted alkyl, R^{6B}-substituted or unsubstituted heteroalkyl, R^{6B}-substituted or unsubstituted cycloalkyl, R^{6B}-substituted or unsubstituted heterocycloalkyl, R^{6B}-substituted or unsubstituted aryl, or 10 R^{6B}-substituted or unsubstituted heteroaryl. R^{6B} is independently halogen, —CN, —CF₃, —CCl₃, —OH, —NH₂, —SO₂, —COOH, oxo, nitro, —SH, —CONH₂, R^{6C}-substituted or unsubstituted alkyl, R^{6C}-substituted or unsubstituted heteroalkyl, R^{6C}-substituted or unsubstituted cycloalkyl, R^{6C}-substituted or unsubstituted heterocycloalkyl, R^{6C}-substituted or unsubstituted aryl, or R^{6C}-substituted or unsubstituted heteroaryl. R^{6C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁷ is hydrogen, or R^{7A}-substituted or unsubstituted alkyl. R^{7A} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

Further to this embodiment, R⁸ is independently halogen, —CN, —SH, —OH, —COOH, —NH₂, —CONH₂, nitro, —CF₃, —CCl₃, R^{8A}-substituted or unsubstituted alkyl, R^{8A}-substituted or unsubstituted heteroalkyl, R^{8A} substituted or unsubstituted cycloalkyl, R^{8A}-substituted or unsubstituted heterocycloalkyl, R^{8A} substituted or unsubstituted aryl, or R^{8A}-substituted or unsubstituted heteroaryl. R^{8A} is independently halogen, —CN, —CF₃, —CCl₃, —OH, —NH₂, —SO₂, —COOH, oxo, nitro, —SH, —CONH₂, R^{8B}-substituted or unsubstituted alkyl, R^{8B}-substituted or unsubstituted heteroalkyl, R^{8B}-substituted or unsubstituted cycloalkyl, R^{8B}-substituted or unsubstituted heterocycloalkyl, R^{8B}-substituted or unsubstituted aryl, or R^{8B}-substituted or unsubstituted heteroaryl. R^{8B} is independently halogen, —CN, —CF₃, —CCl₃, —OH, —NH₂, —SO₂, —COOH, oxo, nitro, —SH, —CONH₂, R^{8C}-substituted or unsubstituted alkyl, 8^{4C}-substituted or unsubstituted heteroalkyl, R^{8C}-substituted or unsubstituted cycloalkyl, R^{8C}-substituted or unsubstituted heterocycloalkyl, R^{8C}-substituted or unsubstituted aryl, or R^{8C}-substituted or unsubstituted heteroaryl. R^{8C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In another aspect, there is provided a compound of formula (II) as disclosed above, provided, however, that: (i) the compound of formula (II) is not

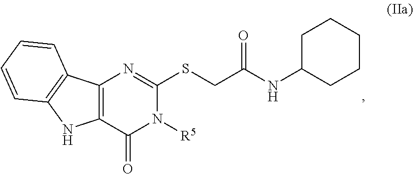

wherein R⁵ is p-fluorophenyl or p-methylphenyl; (ii) the compound is not

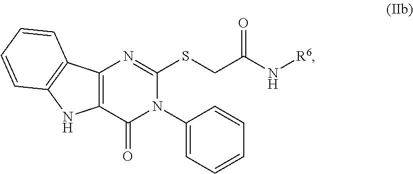

wherein R⁶ is unsubstituted aryl, unsubstituted cyclohexyl, unsubstituted thiazole, or —CH₂-furanyl; or (iii) R⁷ is not hydrogen.

Further to any aspect disclosed above, in one embodiment, R⁵ is not substituted phenyl. In one embodiment, R⁵ is not p-fluorophenyl or p-methylphenyl.

In one embodiment, the compound does not have the structure of formula (IIa) wherein R⁶ is substituted phenyl. In one embodiment, the compound does not have the structure of formula (IIa) wherein R⁶ is p-fluorophenyl or p-methylphenyl.

Further to any aspect disclosed above, in one embodiment, R⁶ is not substituted or unsubstituted aryl, unsubstituted cyclohexyl, unsubstituted thiazole, or —CH₂-furanyl. In one embodiment, the compound does not have the structure of formula (IIb) wherein R⁶ is substituted or unsubstituted aryl, substituted or unsubstituted cyclohexyl, substituted or unsubstituted thiazole, or alkyl substituted with a substituted or unsubstituted furanyl. In one embodiment, R⁶ is not unsubstituted aryl, unsubstituted cyclohexyl, unsubstituted thiazole, or —CH₂-furanyl.

Further to any aspect disclosed above, in one embodiment R⁵ is substituted or unsubstituted cycloalkyl or substituted or unsubstituted aryl. In one embodiment, R⁵ is unsubstituted cycloalkyl or unsubstituted aryl.

In one embodiment, R⁵ is substituted or unsubstituted C₆-C₈ cycloalkyl or substituted or unsubstituted phenyl. In one embodiment, R⁵ is substituted or unsubstituted C₆, cycloalkyl or substituted or unsubstituted phenyl.

In one embodiment, R⁵ is R^{5A}-substituted or unsubstituted C6 cycloalkyl or R^{5A}-substituted or unsubstituted phenyl, wherein R^{5A} is a halogen. In one embodiment, R⁵ is R^{5A}-substituted or unsubstituted phenyl, wherein R^{5A} is a halogen. In one embodiment, R⁵ is R^{5A}-substituted or unsubstituted phenyl, wherein R^{5A} is a fluoro. In one embodiment, R⁵ is unsubstituted phenyl.

Further to any aspect disclosed above, in one embodiment the compound does not have the structure of Formula (Ib) wherein R⁶ is substituted or unsubstituted aryl, substituted or unsubstituted cyclohexyl, substituted or unsubstituted thiazole, or alkyl substituted with a substituted or unsubstituted furanyl.

In one embodiment, R⁶ is substituted or unsubstituted C₄-C₁₂ cycloalkyl, substituted or unsubstituted C₃-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ branched alkyl, or substituted or unsubstituted phenyl. In one embodiment, R⁶ is R^{6A}-substituted or unsubstituted C₄-C₁₂ cycloalkyl, R^{6A}-substituted or unsubstituted C₄-C₁₂ branched alkyl, or R^{6A}-substituted or unsubstituted phenyl, wherein R^{6A} is halogen. In one embodiment, R⁶ is R^{6A}-substituted or unsubstituted C₄-C₁₂ cycloalkyl, R^{6A-}substituted or unsubstituted C₄-C₁₂ branched alkyl, or R^{6A}-substituted or unsubstituted phenyl, wherein R^{6A} is fluoro. In one embodiment, R⁶ is unsubstituted C₄-C₁₂ cycloalkyl, unsubstituted C₄-C₁₂ branched alkyl, or R^{6A}-substituted or unsubstituted phenyl, wherein R^{6A} is fluoro. In one embodiment, R⁶ is unsubstituted C₆-C₁₂ cycloalkyl, unsubstituted C₄-C₁₂ branched alkyl, or unsubstituted phenyl. In one embodiment, R⁶ is unsubstituted C₆-C₁₀ cycloalkyl. In one embodiment, R⁶ is unsubstituted C₆-C₈ cycloalkyl. In one embodiment, R⁶ is unsubstituted cyclohexyl.

In one embodiment, R⁷ is hydrogen or substituted or unsubstituted alkyl. In one 30 embodiment, R⁷ is hydrogen or unsubstituted alkyl. In one embodiment, R⁷ is hydrogen or unsubstituted C1-C3 alkyl. In one embodiment, R⁷ is hydrogen, methyl or ethyl. In one embodiment, R³ is methyl. In one embodiment, R⁷ is ethyl. In one embodiment, R⁷ is hydrogen.

In one embodiment, z1 is 0, 1, 2, 3, or 4. In one embodiment, z1 is 0 or 1. In one embodiment, z1 is 0. In one embodiment, z1 is 1. In one embodiment, z2 is 0, 1, 2, 3, 4, or 5. In one embodiment, z2 is 1.

In one embodiment, R⁸ is independently substituted or unsubstituted alkyl. In one embodiment, R⁸ independently is substituted alkyl. In one embodiment, R⁸ is independently unsubstituted alkyl. In one embodiment, R⁸ is independently substituted or unsubstituted heteroalkyl. In one embodiment, R⁸ is independently substituted heteroalkyl. In one embodiment, R⁸ is independently unsubstituted heteroalkyl.

Further to any aspect disclosed above, in one embodiment there is provided a compound having formula (III):

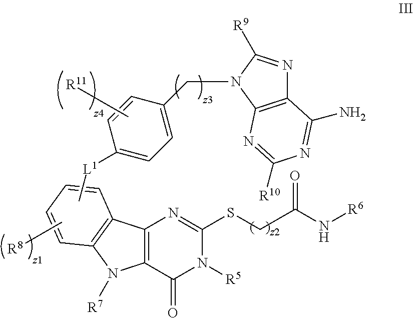

For formula (III), R⁵, R⁶, R⁷, R⁸, z1 and z2 are as disclosed above for formula (II), including embodiments thereof. The symbol z3 is an integer from 1 to 10. The symbol z4 is an integer from 0 to 4. L¹ is substituted or unsubstituted alkylene or substituted or unsubstituted heteroalkylene. R⁹ is —SR^{9A} or —OR^{9A}. R^{9A} is hydrogen, substituted or unsubstituted alkyl, substituted or unsubstituted heteroalkyl, substituted or unsubstituted heterocycloalkyl, substituted or unsubstituted cycloalkyl, substituted or unsubstituted aryl, or substituted or unsubstituted heteroaryl. R¹⁰ is hydrogen, halogen, nitro, —OH, —SH, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In one embodiment, R¹⁰ is R^{10A}-substituted or unsubstituted alkyl, R^{10A} substituted or unsubstituted heteroalkyl, R^{10A} substituted or unsubstituted cycloalkyl, R^{10A} substituted or unsubstituted heterocycloalkyl, R^{10A} substituted or unsubstituted aryl, or R^{10A} substituted or unsubstituted heteroaryl. R^{10A} is independently halogen, —CN, —CF₃, —CCl₃, —OH, —NH₂, —S0₂, —COOH, oxo, nitro, —SH, —CONH₂, R^{10B}-substituted or unsubstituted alkyl, R^{10B}-substituted or unsubstituted heteroalkyl, R^{10B}-substituted or unsubstituted cycloalkyl, R^{10B}-substituted or unsubstituted heterocycloalkyl, R^{10B}-substituted or unsubstituted aryl, or R^{10B}-substituted or 10 unsubstituted heteroaryl. R^{10B} is independently halogen, —CN, —CF₃, —CCl₃, —OH, —NH₂, —SO₂, —COOH, oxo, nitro, —SH, —CONH₂, R^{10C}-substituted or unsubstituted alkyl, R^{10C}-substituted or unsubstituted heteroalkyl, R^{10C}-substituted or unsubstituted cycloalkyl, R^{10C}-substituted or unsubstituted heterocycloalkyl, R^{10C}-substituted or unsubstituted aryl, or R^{10C}-substituted or unsubstituted heteroaryl. R^{10C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In one embodiment, R¹¹ at each occurrence is independently R^{11A}-substituted or unsubstituted alkyl, R^{11A} substituted or unsubstituted heteroalkyl, R^{11A} substituted or unsubstituted cycloalkyl, R^{11A} substituted or unsubstituted heterocycloalkyl, R^{11A} substituted or unsubstituted aryl, or R^{11A} substituted or unsubstituted heteroaryl. R^{11A} is independently halogen, —CN, —CF₃, —CCl₃, —OH, —NH₂, —SO₂, —COOH, oxo, nitro, —SH, —CONH₂, R^{11B}-substituted or unsubstituted alkyl, R^{11B}-substituted or unsubstituted heteroalkyl, R^{11B}-substituted or unsubstituted cycloalkyl, R^{11B}-substituted or unsubstituted heterocycloalkyl, R^{11B}-substituted or unsubstituted aryl, or 25 R^{11B}-substituted or unsubstituted heteroaryl. R^{11B} is independently halogen, —CN, —CF₃, —CCl₃, —OH, —NH₂, —S0₂, —COOH, oxo, nitro, —SH, —CONH₂, R^{11C}-substituted or unsubstituted alkyl, R^{11C}-substituted or unsubstituted heteroalkyl, R^{11C}-substituted or unsubstituted cycloalkyl, R^{11C}-substituted or unsubstituted heterocycloalkyl, R^{11C}-substituted or unsubstituted aryl, or R^{11C}-substituted or unsubstituted heteroaryl. R^{11C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In one embodiment, z3 is an integer from 1 to 3. In one embodiment, z3 is 1. In one embodiment, z4 is 0.

In one embodiment, R⁹ is —OH. In one embodiment, R^{9A} is hydrogen.

In one embodiment, L¹ is R¹²-substituted or unsubstituted alkylene, or R′ 2-substituted 5 or unsubstituted heteroalkylene. R¹² is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl. In one embodiment, L¹ is R¹²-substituted alkylene. In one embodiment, L¹ is unsubstituted alkylene. In one embodiment, L¹ is R¹²-substituted heteroalkylene. In one 10 embodiment, L¹ is unsubstituted heteroalkylene. In one embodiment, L¹ is enzymatically cleavable. The terms “enzymatically cleavable” and the like refer, in the usual and customary sense, to a chemical moiety which can undergo bond scission by the action of an enzyme, e.g., hydrolase, esterase, lipase, peptidase, amidase and the like. Scission can occur at a terminal bond of L¹ or a non-terminal bond within L¹. Bond scission of L¹ can be accompanied by bond 15 arrangement of the resulting fragments of L¹ and bond addition, e.g., addition of water (e.g., under the action of a hydrolase, esterase, lipase, peptidase, amidase and the like). Enzymatic cleavage can occur under physiological conditions, e.g., under the action of a physiological enzyme within an organism. Enzymatic cleavage can occur within a cell, e.g., a biological cell as disclosed herein. Enzymatic cleavage can occur extracellularly, e.g., in the circulatory system 20 of a subject. Enzymatic cleavage can occur under in vitro conditions.

In one embodiment, L¹ is —C(0)-X³-L^{1A}-X⁴—C(0)-, wherein X³ and X⁴ are —O— or —NH—, and L^{1A} is substituted or unsubstituted alkylene or substituted or unsubstituted heteroalkylene. In one embodiment, L^{1A} is -L^{1B}-(CH₂CH₂₀)ₙ— wherein n is an integer from 1 to 100, and L^{1B} is unsubstituted C₁-C₁₀ alkylene. In one embodiment, n is an integer in the range of about 1 to 100, 1 to 90, 1 to 80, 1 to 70, 1 to 60, 1 to 50, 1 to 40, 1 to 30, 1 to 20, or 1 to 10. In one embodiment, n is about 100, 90, 80, 70, 60, 50, 40, 30, 20, 18, 16, 14, 12, 10, or 9, 8, 7, 6, 5, 4, 3, or 2. In one embodiment, n is an integer in the range of about 1 to 100, 1 to 90, 1 to 80, 1 to 70, 1 to 60, 1 to 50, 1 to 40, 1 to 30, 1 to 20, or 1 to 10, and L^{1B} is ethylene. In one embodiment, n is an integer from 1 to 10, and L^{1B} is ethylene. In one embodiment, L¹ is 30 —C(0)0-CH₂CH₂-(0CH₂CH₂)n-NH—C(0)-, wherein n is 1 to 10, e.g., 1, 2, 3, 4, 5, 6, 7, 8, 9, or 10.

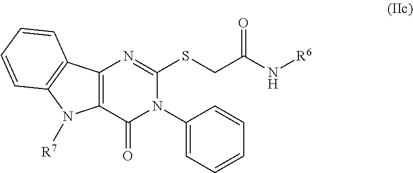

For formula (IIc) (above), R⁶ is substituted or unsubstituted alkyl, or substituted or unsubstituted cycloalkyl; and R⁷ is substituted or unsubstituted alkyl. In one embodiment, R⁶ is unsubstituted cycloalkyl, e.g., cyclohexyl, cycloheptyl or cyclooctyl. In one embodiment, R⁶ is unsubstituted alkyl, e.g., 3,3-dimethylbutyl. In one embodiment, R⁷ is unsubstituted alkyl. In one embodiment, R¹⁰ is an alkyl ester.

In another aspect, there is provided a compound having formula (IId):

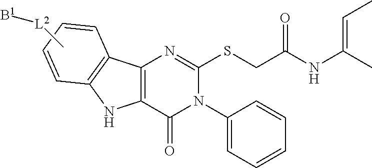

For formula (IId), L² is a linker, and B¹ is a purine base or analog thereof.

In one embodiment, L² is a substituted or unsubstituted alkylene, or a substituted or unsubstituted heteroalkylene. In one embodiment, L² includes a water soluble polymer. A “water soluble polymer” means a polymer which is sufficiently soluble in water under physiologic conditions of e.g., temperature, ionic concentration and the like, as known in the art, to be useful for the methods described herein. An exemplary water soluble polymer is polyethylene glycol. In one embodiment, the water soluble polymer is -(0CH₂CH₂)ₘ— wherein m is 1 to 100. In one embodiment, L² includes a cleavage element. A “cleavage element” is a chemical functionality which can undergo cleavage (e.g., hydrolysis) to release the compound, optionally including remnants of linker L², and B¹, optionally including remnants 20 of linker L².

A representative schematic synthesis is depicted in Scheme 4 following, wherein element (i) is a modified versatile intermediate TLR4 ligand, element (ii) is a TLR7 ligand with linker, and element (iii) is a TLR4-TLR7 dual ligand conjugate (cleavable linkage shown). Methods of conjugation of elements (i) and (ii) are well known in the art to afford the resulting dual ligand conjugate.

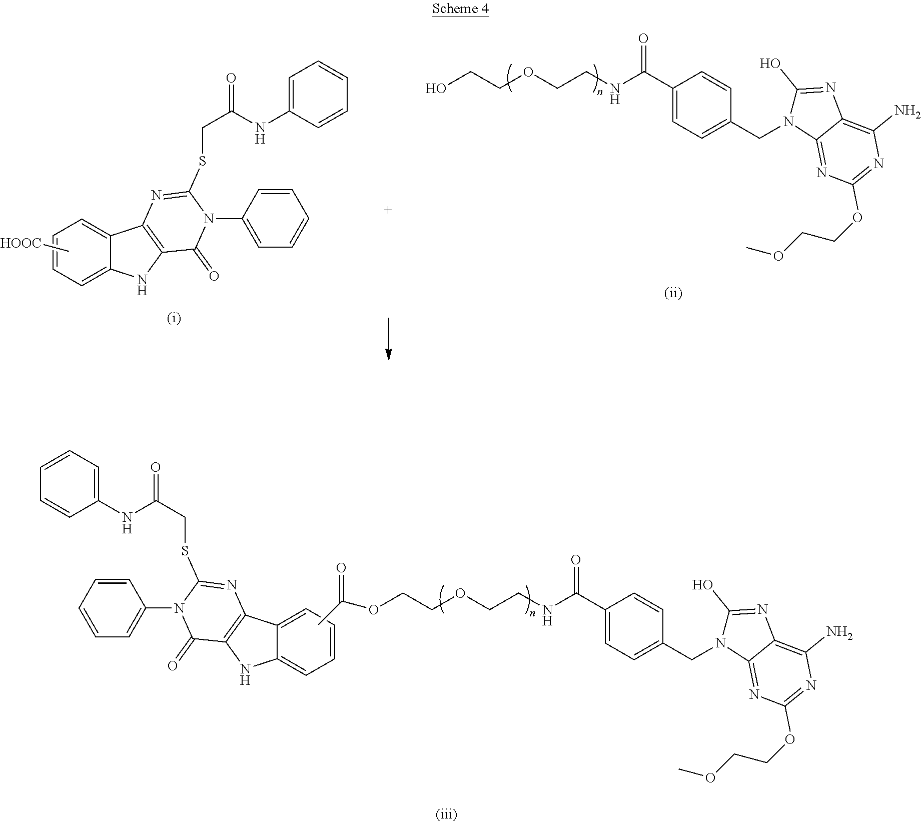

In another aspect, there is provided a compound having formula (IIe):

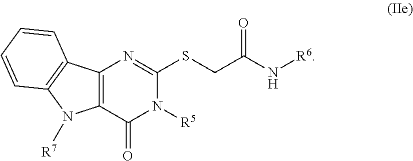

For formula (IIe), R⁶ and R⁷ are as disclosed for formula (III). R⁵ is substituted or unsubstituted alkyl, substituted or unsubstituted heteroalkyl, substituted or unsubstitutedcycloalkyl, substituted or unsubstituted heterocycloalkyl, substituted or unsubstituted aryl, and substituted or unsubstituted heteroaryl.

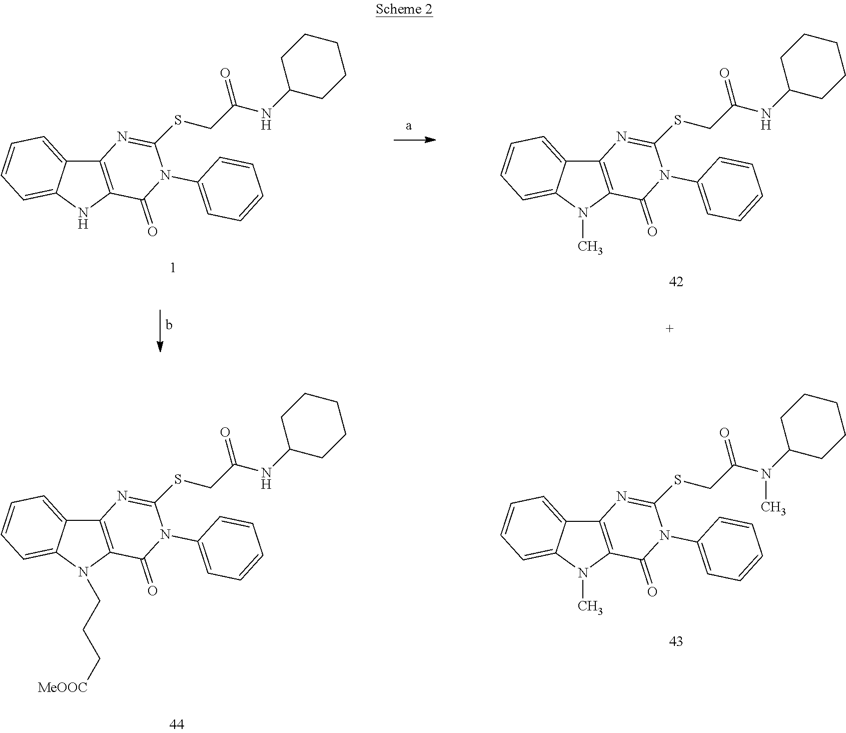 [SMILES: O=C(CSc1nc2c([nH]c3ccccc32)c(=O)n1-c1ccccc1)NC1CCCCC1 ; Cn1c2ccccc2c2nc(SCC(=O)NC3CCCCC3)n(-c3ccccc3)c(=O)c21 ; COC(=O)CCCn1c2ccccc2c2nc(SCC(=O)NC3CCCCC3)n(-c3ccccc3)c(=O)c21 ; CN(C(=O)CSc1nc2c3ccccc3n(C)c2c(=O)n1-c1ccccc1)C1CCCCC1]

Tables 1, 2 and 3 provide exemplary TLR4 ligands and their I-6, IL-8 and IP-10 activity.

TABLE 1

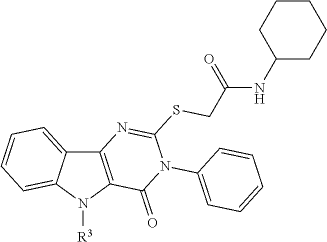

| | | mouse | | | human | |
|---|---|---|---|---|---|---|
| Compound | R³ | IL-6^{a} | IP-10^{b} | TLR4^{a} | IL-8^{c} | TLR4^{c} |
| 1 | H | 100 | 100 | 100 | 100 | 100 |
| 42 | CH₃ | 101 | 99 | 100 | 79 | 92 |
| 43 | CH₃, N-methyl | <1 | <1 | 1 | 5 | <1 |
| 44 | 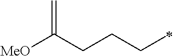 | 4 | 19 | 9 | 124 | 19 |
| 48 | 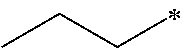 | 49 | 45 | 43 | 83 | 117 |
| 49 | 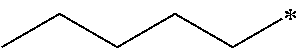 | 5 | <1 | 6 | 1 | 11 |
| 50 | 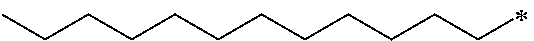 | <1 | <1 | 4 | <1 | 10 |
| 51 | 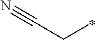 | 98 | 64 | 55 | 30 | 19 |
| 52 | 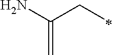 | 1 | 1 | 8 | 4 | 11 |

TABLE 2

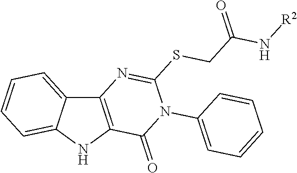

| Com- | | mouse | | | human | |
|---|---|---|---|---|---|---|
| pound | R² | IL-6^{a} | IP-10^{b} | TLR4^{c} | IL-8^{d} | TLR4^{e} |
| 1 | 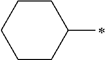 | 100 | 100 | 100 | 100 | 100 |
| 9 | 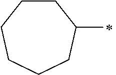 | 126 | 107 | 99 | 118 | 100 |
| 10 | 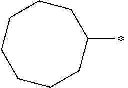 | 114 | 98 | 95 | 73 | 64 |
| 11 | 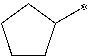 | 53 | 52 | 28 | 5 | 23 |
| 12 | 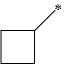 | 35 | 66 | 18 | 19 | 32 |
| 13 | 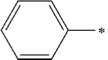 | 11 | 44 | 21 | 15 | 31 |
| 14 | 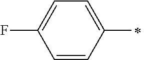 | 103 | 70 | 81 | 107 | 108 |
| 15 | 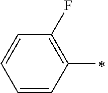 | 36 | 54 | 43 | 61 | 71 |
| 16 | 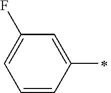 | 14 | 45 | 11 | 13 | 39 |
| 17 | 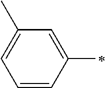 | 19 | 55 | 13 | 54 | 116 |
| 18 | 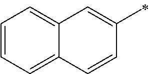 | <1 | <1 | 2 | <1 | 21 |
| 19 | 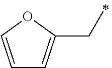 | 18 | 26 | 5 | 15 | 27 |
| 20 | 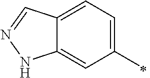 | 3 | <1 | <1 | 7 | <1 |
| 21 | 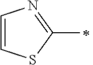 | 1 | <1 | 2 | 5 | 12 |
| 22 | 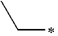 | <1 | <1 | <1 | 4 | 8 |
| 23 | 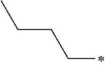 | 49 | 61 | 31 | 29 | 32 |
| 24 | 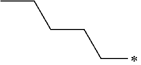 | 40 | 40 | 42 | 25 | 23 |
| 25 | 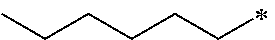 | <1 | <1 | 4 | 7 | 8 |
| 26 | 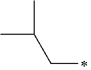 | 44 | 53 | 24 | 11 | 21 |
| 27 | 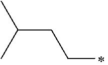 | 61 | 62 | 56 | 53 | 38 |
| 28 | 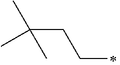 | 99 | 85 | 126 | 95 | 72 |
| 29 | 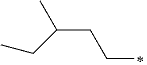 | 30 | 45 | 29 | 23 | 17 |
| 30 | 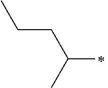 | 12 | 55 | 17 | 6 | 13 |
| 31 | 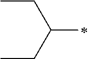 | 10 | 26 | 8 | <1 | 11 |
| 32 | 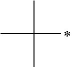 | <1 | <1 | <1 | 5 | 6 |
| 33 | 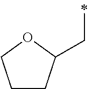 | 23 | 54 | 21 | 8 | 13 |
| 34 | 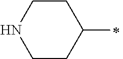 | <1 | <1 | <1 | 17 | <1 |
| 35 | DOPE | <1 | <1 | <1 | <1 | <1 |

TABLE 3

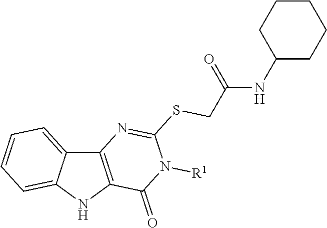

| Com- | | mouse | | | human | |
|---|---|---|---|---|---|---|
| pound | R¹ | IL-6^{a} | IP-10^{b} | TLR4^{c} | IL-8^{d} | TLR4^{e} |
| 1 | 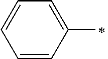 | 100 | 100 | 100 | 100 | 100 |
| 36 | 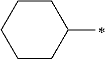 | 71 | 61 | 56 | 27 | 40 |
| 37 | 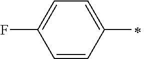 | 48 | 72 | 41 | 5 | 51 |
| 38 | 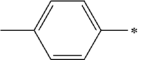 | <1 | <1 | 2 | 6 | 2 |
| 39 | 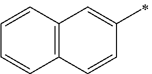 | 3 | <1 | 6 | <1 | 3 |
| 40 | 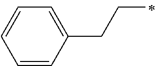 | <1 | <1 | 1 | 5 | 1 |
| 41 | 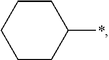 | 47 | 47 | 19 | 14 | 33 |
| | N-cyclopentyl | | | | | |

### Additional Conjugates for Use in the Methods

The TLR conjugates may include a homofunctional TLR ligand. For example, the TLR7 agonist can be a 7-thia-8-oxoguanosinyl (TOG) moiety, a 7-deazaguanosinyl (7DG) moiety, a resiquimod moiety, or an imiquimod moiety. In another embodiment, the TLR agonist conjugate may include a heterofunctional TLR agonist polymer. The heterofunctional TLR agonist polymer may include a TLR7 agonist and a TLR4 agonist.

In one embodiment, the invention provides the following conjugates

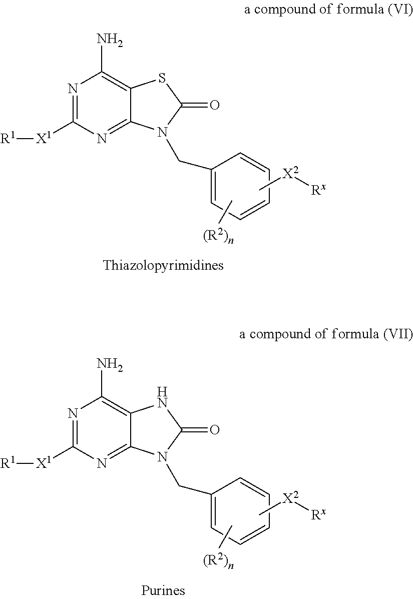

X¹═—O—,—S—, or —NR^{c}—,

wherein R^{c} hydrogen, C₁₋₁₀alkyl, or C₁₋₁₀alkyl substituted by C₃₋₆ cycloalkyl, or R^{c} and R¹ taken together with the nitrogen atom can form a heterocyclic ring or a substituted heterocyclic ring, wherein the substituents are hydroxy, C₁₋₆ alkyl, hydroxy C₁₋₆ alkylene, C₁₋₆ alkoxy, C₁₋₆ alkoxy C₁₋₆ alkylene, or cyano;

wherein R¹ is (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic; wherein the substituents on the alkyl, aryl or heterocyclic groups are hydroxy, C₁₋₆ alkyl, hydroxy C₁₋₆ alkylene, C₁₋₆ alkoxy, C₁₋₆ alkoxy C₁₋₆ alkylene, amino, cyano, halogen, or aryl;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, —(C₁-C₆)alkylene-NR^{a}R^{b}, —(C₁-C₆)alkylene-C(O)NR^{a}R^{b}, halo, nitro, or cyano;

wherein each R^{a} and R^{b} is independently hydrogen, (C₁₋₆)alkyl, (C₃-C₅)cycloalky, (C₁₋₆6)alkoxy, halo(C₁₋₆)alkyl, (C₃-C₈)cycloalkyl(C₁₋₆)alkyl, (C₁₋₆)alkanoyl, hydroxy(C₁₋₆)alkyl, aryl, aryl(C₁₋₆)alkyl, aryl, aryl(C₁₋₆)alkyl, Het, Het (C₁₋₆)alkyl, or (C₁₋₆)alkoxycarbonyl; wherein X² is a bond or a linking group; wherein R^{x} is an auxiliary group such as a macromolecule, wherein n is 0, 1, 2, 3, or 4; or a tautomer thereof, or a pharmaceutically acceptable salt thereof.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g., ethylene: —CH₂—CH₂—).

C₃₋₇ Cycloalkyl includes groups such as, cyclopropyl, cyclopentyl, cyclohexyl, cycloheptyl, and the like, and alkyl-substituted C₃₋₇ cycloalkyl group, e.g., straight or branched C₁₋₆ alkyl group such as methyl, ethyl, propyl, butyl or pentyl, and C₅₋₇ cycloalkyl group such as, cyclopentyl or cyclohexyl, and the like.

Lower alkoxy includes C₁₋₆ alkoxy groups, such as methoxy, ethoxy or propoxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl;

Lower alkoxycarbonyl includes C₂₋₇ alkoxycarbonyl groups, such as methoxycarbonyl, ethoxycarbonyl or propoxycarbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g., dimethylamino, diethylamino, ethylmethylamino).

Lower alkylcarbamoyl group means carbamoyl group substituted by C₁₋₆ alkyl group (e.g., methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g., dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic or heterocycle refers to monocyclic saturated heterocyclic groups, or unsaturated monocyclic or fused heterocyclic group containing at least one heteroatom, e.g., 0-3 nitrogen atoms NR^{c}, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofuranyl, and the like. A Het group can be a saturated heterocyclic group or an unsaturated heterocyclic group, such as a heteroaryl group.

R^{c} and R¹ taken together with the nitrogen atom to which they are attached can form a heterocyclic ring. Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl, and the like.

The alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano. A specific value for X³ is a sulfur atom, an oxygen atom or —NR^{d}—.

Another specific X¹ is a sulfur atom.

Another specific X¹ is an oxygen atom.

Another specific X¹ is —NR^{c}—.

Another specific X¹ is —NH—.

A specific value for R^{c} is hydrogen, C₁₋₄ alkyl or substituted C₁₋₄ alkyl.

A specific value for R¹ and R^{c} taken together is when they form a heterocyclic ring or a substituted heterocyclic ring.

Another specific value for R¹ and R^{c} taken together is substituted or unsubstituted morpholino, piperidino, pyrrolidino, or piperazino ring

A specific value for R¹ is hydrogen, C₁₋₄alkyl, or substituted C₁₋₄alkyl.

Another specific R¹ is 2-hydroxyethyl, 3-hydroxypropyl, 4-hydroxybutyl, 2-aminoethyl, 3-aminopropyl, 4-aminobutyl, methoxymethyl, 2-methoxyethyl, 3-methoxypropyl, ethoxymethyl, 2-ethoxyethyl, methylthiomethyl, 2-methylthioethyl, 3-methylthiopropyl, 2-fluoroethyl, 3-fluoropropyl, 2,2,2-trifluoroethyl, cyanomethyl, 2-cyanoethyl, 3-cyanopropyl, methoxycarbonylmethyl, 2-methoxycarbonylethyl, 3-methoxycarbonylpropyl, benzyl, phenethyl, 4-pyridylmethyl, cyclohexylmethyl, 2-thienylmethyl, 4-methoxyphenylmethyl, 4-hydroxyphenylmethyl, 4-fluorophenylmethyl, or 4-chlorophenylmethyl.

Another specific R¹ is hydrogen, CH₃—, CH₃—CH₂—, CH₃CH₂CH₂—, hydroxyC₁₋₄alkylene, or C₁₋₄alkoxyC₁₋₄alkylene.

Another specific value for R¹ is hydrogen, CH₃—, CH₃—CH₂—, CH₃—O—CH₂CH₂— or CH₃—CH₂—O—CH₂CH₂—.

A specific value for R² is halogen or C₁₋₄alkyl.

Another specific value for R² is chloro, bromo, CH₃—, or CH₃—CH₂—.

Specific substituents for substitution on the alkyl, aryl or heterocyclic groups are hydroxy, C₁₋₆alkyl, hydroxyC₁₋₆alkylene, C₁₋₆alkoxy, C₁₋₆alkoxyC₁₋₆alkylene, C₃₋₆cycloalkyl, amino, cyano, halogen, or aryl.

A specific value for X² is a bond or a chain having up to about 24 atoms; wherein the atoms are selected from the group consisting of carbon, nitrogen, sulfur, non-peroxide oxygen, and phosphorous. Any carbon atom can bear an oxo group, and any sulfur atom can bear one or two oxo groups. The chain can be interspersed with one or more cycloalkyl, aryl, heterocyclyl, or heteroaryl rings.

Another specific value for X² is a bond or a chain having from about 4 to about 12 atoms.

Another specific value for X² is a bond or a chain having from about 6 to about 9 atoms.

Another specific value for X² is a carbonyl (C(O)) group.

Certain non-limiting examples of X² include —(Y)_{y}—, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C₁₋₆ alkoxy C₁₋₆ alkenyl, amino, cyano, halogen or aryl. In certain embodiments, a linker sometimes is a —C(Y′)(Z′)—C(Y″)(Z″)— linker, where each Y′, Y″, Z′ and Z″ independently is hydrogen C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C₁₋₆ alkoxy C₁₋₆ alkenyl, amino, cyano, halogen or aryl.

Another specific value for X² is

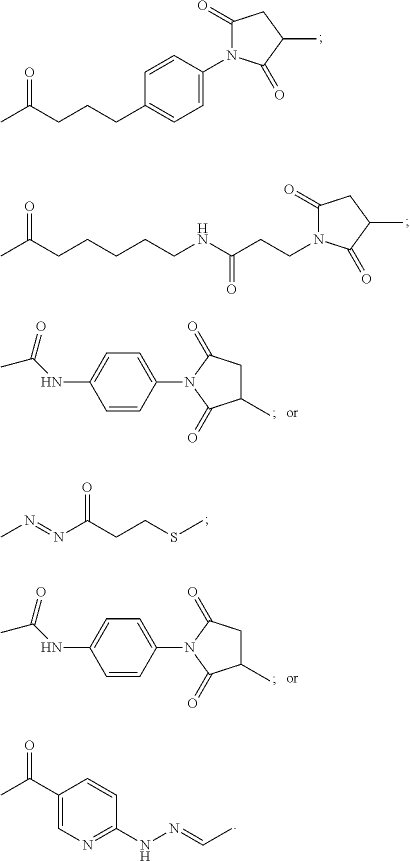

Another specific value for X⁴ is

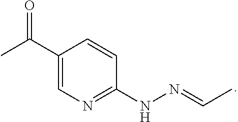

The invention includes compositions that include one or more synthetic TLR4 agonists, synthetic TLR7 agonists, or a combination thereof of the invention. Other non-limiting examples are known and are disclosed in U.S. published patent application No. 20050004144.

### Routes and Formulations

Administration of compositions having one or more antigens and one or more adjuvants of the invention and another active agent or administration of a composition having one or more antigens and a composition having one or more adjuvants, can be via any of suitable route of administration, particularly parenterally, for example, intravenously, intra-arterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

The compositions invention alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent, e.g., an antigen, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound may also be administered intravenously or intraperitoneally by infusion or injection. Solutions of the antigen(s), and adjuvant(s) optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the antigen(s) and adjuvant(s) optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations of the antigen(s) and adjuvant(s) optionally in combination with another active compound for inhalation delivery. For example, formulations may be designed for aerosol use in devices such as metered-dose inhalers, dry powder inhalers and nebulizers.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949. The ability of an adjuvant to act as a TLR agonist may be determined using pharmacological models which are well known to the art, including the procedures disclosed by Lee et al., Proc. Natl. Acad. Sci. USA, 100: 6646 (2003).

Generally, the concentration of the phospholipid conjugate optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the antigen(s) and adjuvant(s) optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The antigen(s) and adjuvant(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and increased depending on the patient's global response.

The invention will be described by the following non-limiting example.

### EXAMPLE

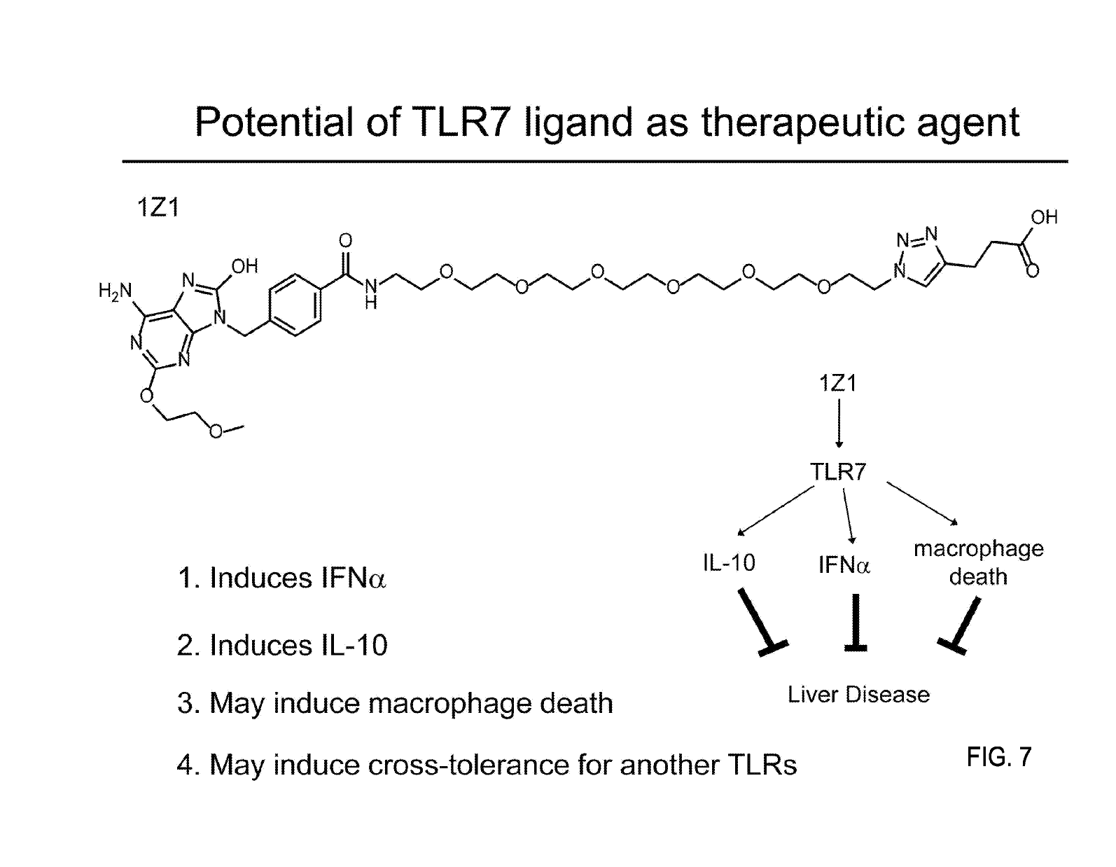

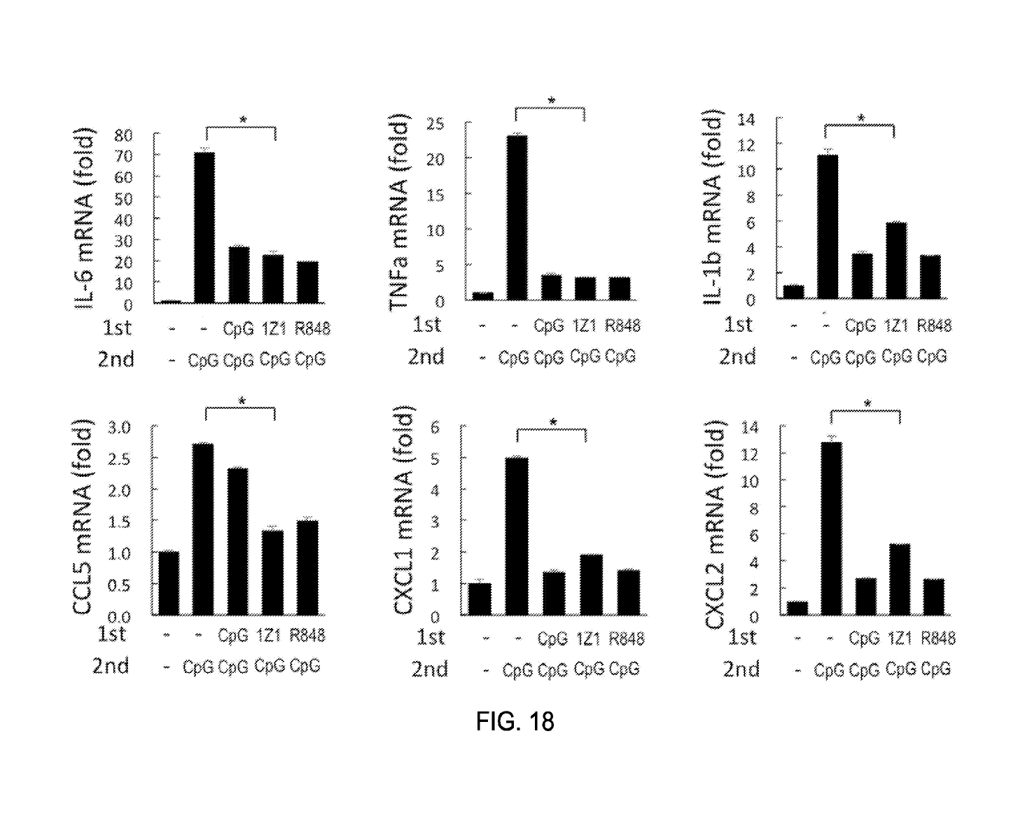

It was examined whether 1Z1 (FIG. 7) inhibits TLR9 signaling, which promotes liver diseases, including non-alcoholic steatohepatitis (NASH) and alcoholic liver disease (ALD). Isolated Kupffer cells were treated with 1Z1 (5 μM) or R848 (0.5 μM) for 16 hours followed by stimulation with CpG-DNA (TLR9 ligand; 5 μg/mL) for 4 hours and inflammatory cytokine expression was then measured. Pretreatment with CpG-DNA, 1Z1, and R848 inhibited production of IL-6, TNFα, IL-1β, CCL5, CXCL1, and CXCL2 in Kupffer cells following CpG-DNA treatment (FIG. 18). This result indicates that 1Z1 can suppress TLR9-mediated inflammatory response.*p<0.05.

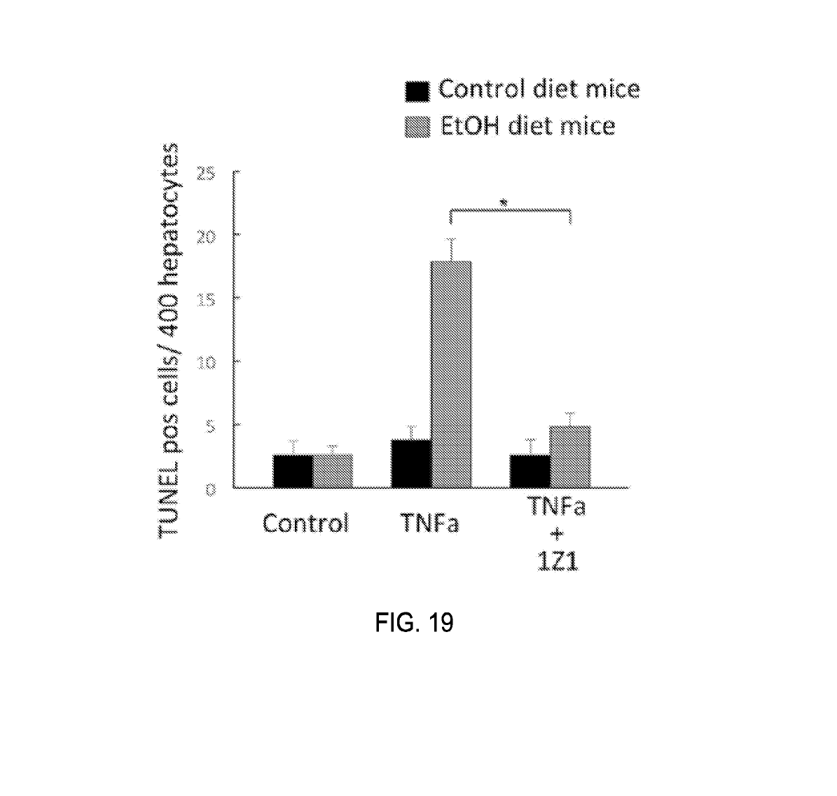

It was also examined whether 1Z1 had a protective effect on hepatocyte death, a major alcohol-induced feature in hepatocytes. Lipid-accumulated hepatocytes isolated from ethanol-treated mice are susceptible to TNFα-mediated cell death. Hepatocytes were isolated from ethanol diet-fed WT mice and treated with TNFα (5 ng/mL) for 24 hours. TNFα induced death in these hepatocytes whereas 1Z1 (5 μM)-treated cells were prevented from TNFα-mediated death (FIG. 19). Thus, 1Z1 has a protective effect on ethanol and TNFα-mediated hepatocyte death.

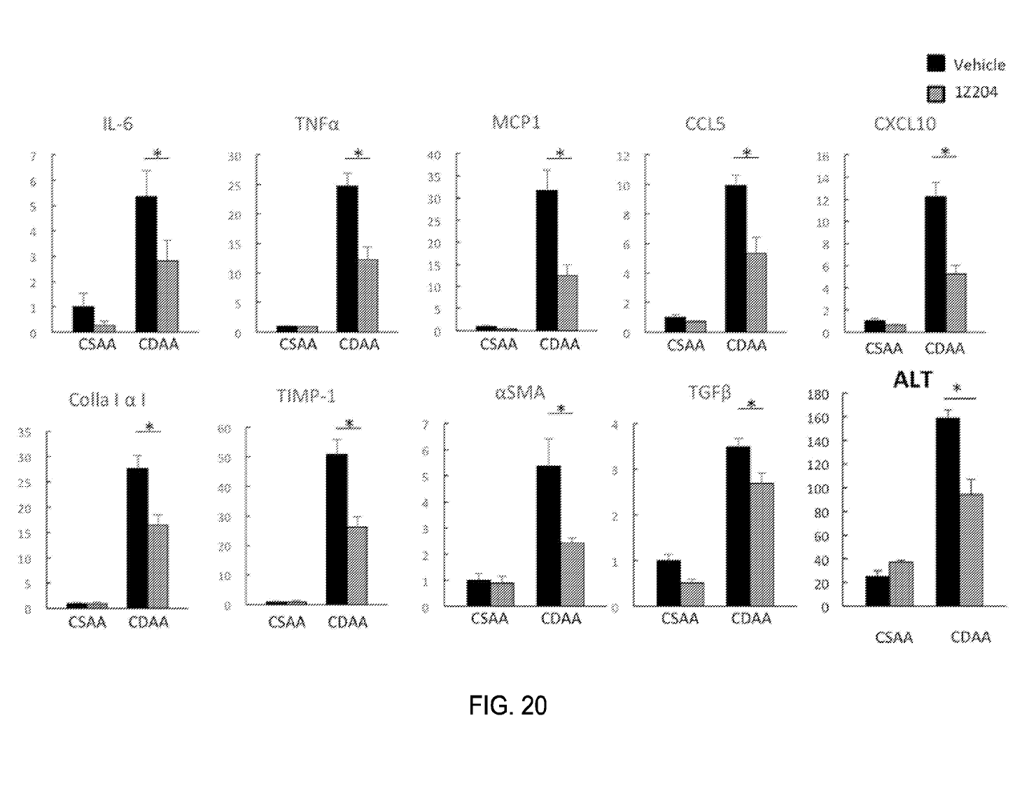

WT mice were fed a choline-sufficient control (CSAA) diet or CDAA diet for 3 weeks. During the last 2 weeks, mice were treated with Vehicle or 0.5 μmol of 1Z204 (TLR4 agonist) IP every other day. qPCR analysis was performed for mRNA expression of IL-6, TNFα, MCP-1, CCL5, CXCL10, collagen α1(I), TIMP-1, αSMA, and TGF-β and serum ALT levels were measured. Inflammatory cytokines IL-6, TNFα, MCP-1, CCL5, CXCL10 and fibrogenic markers collagen al(I), TIMP-1, αSMA, and TGF-β were increased in Vehicle-treated CDAA diet-fed mice, whereas 1Z204 treatment suppressed levels of these cytokines and fibrogenic markers (FIG. 20). Increased ALT levels were also significantly suppressed in the mice treated with 1Z204. These results indicate that 1Z204 has protective effects in a mouse NASH model. n=8 in each group.

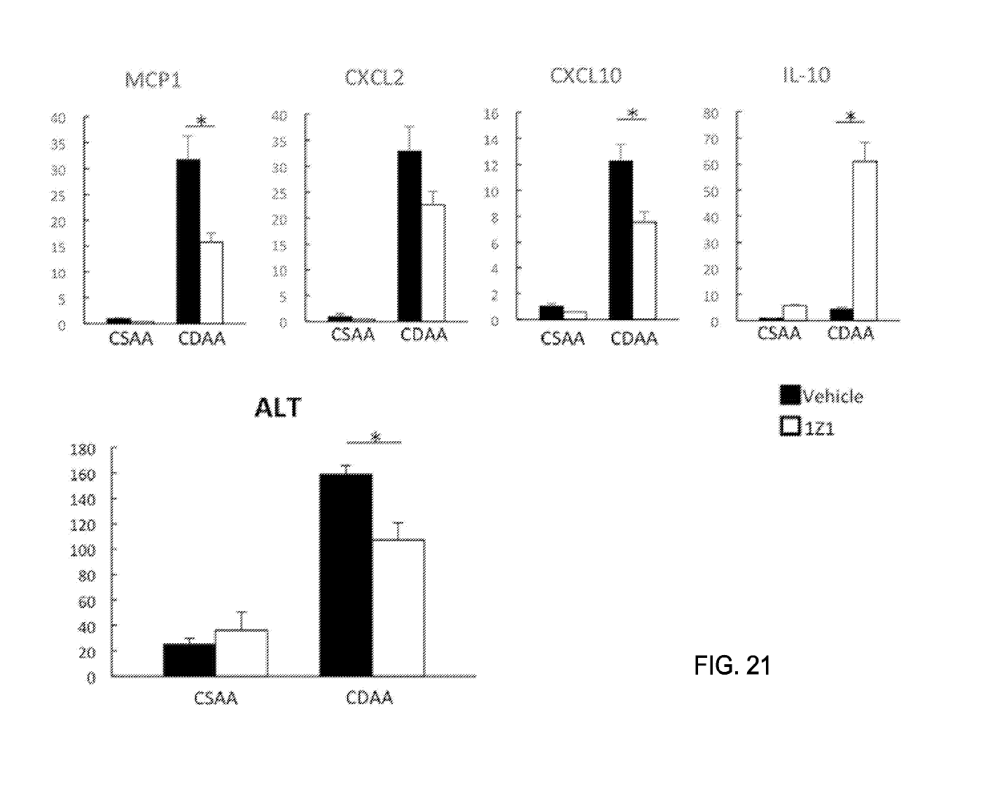

WT mice were fed a choline-sufficient control (CSAA) diet or CDAA diet for 3 weeks. During the last 2 weeks, mice were treated with Vehicle or 0.4 μmol of 1Z1 (TLR7 agonist) SC every day. qPCR analysis was performed for mRNA expression of MCP-1, CXCL2, CXCL10, and IL-10, and serum ALT levels were measured. Inflammatory cytokines MCP-1, CXCL2, and CXCL10 were increased in Vehicle-treated CDAA diet-fed mice, whereas 1Z1 treatment suppressed levels of these chemokines. Notably, 1Z1 significantly upregulated anti-inflammatory IL-10 expression in the liver (FIG. 21). Increased ALT levels were significantly suppressed in the mice treated with 1Z204. These results suggest that 1Z1 has protective effects in a mouse NASH model through production of IL-10. n=8 in each group.

The data support the view that 1Z1 (a TLR7 ligand with attenuated agonist activity) can be useful to tolerize or protect against liver damage caused by inflammatory responses induced in a variety of ways (diet, ethanol, other TLR agonists, etc.). 1Z1 (a TLR4 weak agonist) appears to show similar protective effects, at least in the NASH model.

In summary TLR4 promotes and TLR7 inhibits liver disease (liver fibrosis, alcoholic liver disease, non-alcoholic steatohepatitis). A TLR4 ligand 1Z204 suppressed mouse models of ALD and NASH, which may be mediated by the predominant activation of TRIF pathway. A TLR7 ligand 1Z1 suppressed mouse model of ALD and NASH, which may be through induction of IL-10, IFNα, or macrophage depletion.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit or treat liver disease in a mammal, comprising administering to the mammal an effective amount of a composition comprising a compound of formula (I), a composition comprising a compound of formula (II), or a composition comprising a compound of formula (I) and a compound of formula (II),
wherein formula (I) is 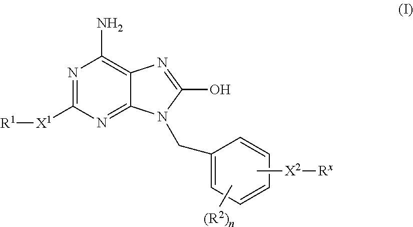
wherein X¹ is —O—, —S—, or —NR^{c}—;
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, or substituted C₅₋₉heterocyclic;
R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;
X² is a bond or a linking group; and
R^{X} is an auxiliary group that is optionally —((R³)ᵣ—(R⁴)ₛ)ₚ, wherein each R³ independently is a polyethylene glycol (PEG) moiety; wherein each R⁴ independently is H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic; wherein r is 1 to 1000, wherein s is 0 to 100, and wherein p is 1 to 100;
or a tautomer thereof;
or a pharmaceutically acceptable salt or solvate thereof;
and wherein formula (II) is 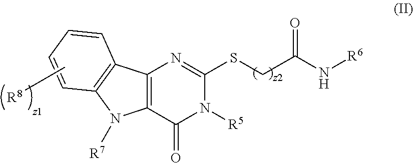
wherein z1 is an integer from 0 to 4, and z2 is an integer from 0 to 5, R⁵ is substituted or unsubstituted cycloalkyl, substituted or unsubstituted heterocycloalkyl, substituted or unsubstituted aryl, or substituted or unsubstituted heteroaryl, R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, R⁷ is hydrogen, or substituted or unsubstituted alkyl, and R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
or a tautomer thereof;
or a pharmaceutically acceptable salt or solvate thereof.

2. The method of claim 1, wherein X² is C(O),

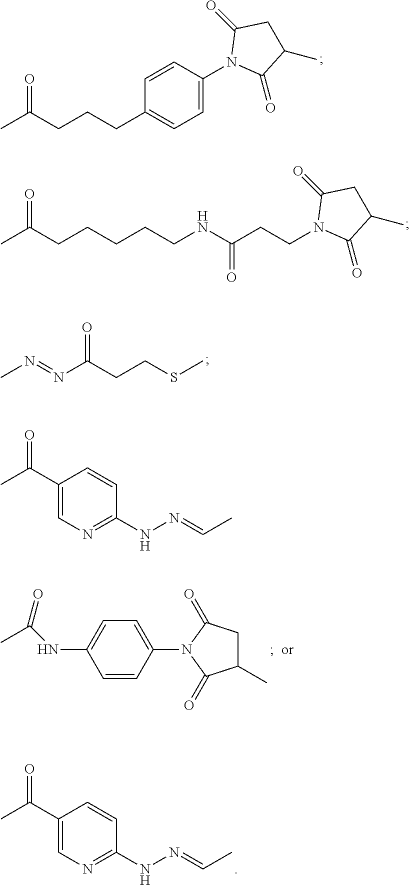

3. The method of claim 1, wherein X¹ is —O—; or wherein X¹ is —S—, or —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl; or wherein X¹ is —NH—.

4-5. (canceled)

6. The method of claim 2, wherein R¹ and R^{c} taken together form a heterocyclic ring or a substituted heterocyclic ring.

7. The method of claim 6, wherein R¹ and R^{c} taken together form a substituted or unsubstituted morpholino, piperidino, pyrrolidino, or piperazino ring.

8. The method of claim 1, wherein R¹ is a C₁-C₁₀ alkyl substituted with C₁₋₆ alkoxy; or wherein R¹ is hydrogen, C₁₋₄alkyl, or substituted C₁₋₄alkyl; or wherein R¹ is hydrogen, methyl, ethyl, propyl, butyl, hydroxyC₁₋₄alkylene, or C₁₋₄alkoxyC₁₋₄alkylene; or wherein R¹ is hydrogen, methyl, ethyl, methoxyethyl, or ethoxyethyl.

9-11. (canceled)

12. The method of claim 1, wherein R² is halo or C₁₋₄alkyl, or R² is absent or wherein R² is chloro, bromo, methyl, or ethyl, or R² is absent.

13. (canceled)

14. The method of claim 1, wherein X¹ is O, R¹ is C₁₋₄alkoxy-ethyl, n is 0, and X² is carbonyl.

15. The method of claim 1, wherein R^{x} is a PEG moiety.

16. The method of claim 15, wherein the PEG moiety comprises about 1 to about 1,000 PEG units.

17. The method of claim 15, wherein one or more of the PEG moieties are linear.

18. The method of claim 15, wherein each PEG unit is —O—CH₂—CH₂— or —CH₂—CH₂—O—.

19. The method of claim 1, wherein r is about 15 to about 500.

20. The method of claim 15, wherein one or more of the PEG moieties are branched.

21. The method of claim 1, wherein the mammal is a human.

22. The method of claim 1, wherein the composition is intranasally, orally or parenterally administered.

23. (canceled)

24. The method of claim 1, wherein formula (II) is not: 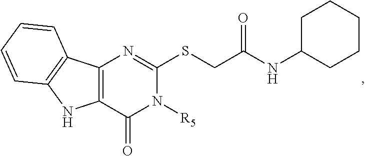
wherein R⁵ is p-fluorophenyl or p-methylphenyl; or is not 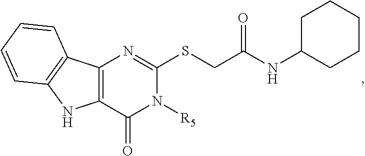
wherein R⁵ is substituted phenyl or wherein R⁵ is not p-fluorophenyl or p-methylphenyl or is not substituted phenyl or wherein R⁶ is not substituted or unsubstituted aryl, unsubstituted cyclohexyl, unsubstituted thiazole, or —CH₂-furanyl
or wherein formula (II) is not 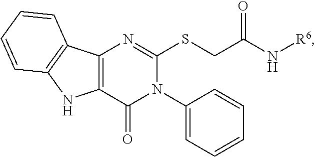
wherein R⁶ is unsubstituted aryl, unsubstituted cyclohexyl, unsubstituted thiazole, or —CH₂— furanyl or is not 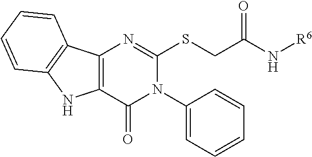
wherein R⁶ is substituted or unsubstituted aryl, substituted or unsubstituted cyclohexyl, substituted or unsubstituted thiazole, or alkyl substituted with a substituted or unsubstituted furanyl.

25-27. (canceled)

28. The method of claim 1, wherein R⁵ is substituted or unsubstituted cycloalkyl or substituted or unsubstituted aryl or wherein R⁶ is substituted or unsubstituted C₃-C₁₂ alkyl, or substituted or unsubstituted aryl or wherein R⁷ is hydrogen or substituted or unsubstituted C₁-C₆ alkyl.

29-30. (canceled)

31. The method of claim 1, wherein z1 is 0.

32-33. (canceled)

34. The method of claim 24, wherein z1 is 0, z2 is 1, R⁵ is substituted or unsubstituted aryl; and R⁶ and R⁷ are independently a substituted or unsubstituted (C₁-C₁₀)alkyl.
